(19) United States
(12) Patent Application Publication
(10) Pub. No.: US 2026/0330989 A1
(43) Pub. Date: Oct. 22, 2026

(54) MIRROR SPEECH ENTRAINMENT FOR SPEECH THERAPY

(71) Applicant: UNIVERSITY OF SOUTH FLORIDA, Tampa, FL (US)

(72) Inventors: Gerald IMAEZUE, Tampa, FL (US); Celine DAVIS, Tampa, FL (US)

(21) Appl. No.: 19/650,279

(22) Filed: Apr. 16, 2026

Related U.S. Application Data
(60) Provisional application No. 63/789,666, filed on Apr. 16, 2025.

(51) Int. Cl. G09B 5/04; A61B 5/00; G06T 13/20; G06T 13/40; G06T 13/80; G10L 13/027; G10L 13/033; G10L 13/08

(52) U.S. Cl. CPC G09B 5/04; A61B 5/4803; A61B 5/741; G06T 13/205; G06T 13/40; G06T 13/80; G10L 13/027; G10L 13/0335; G10L 13/08

(57) ABSTRACT

A method for providing mirror speech entrainment (MSE)-based speech therapy includes obtaining a voice sample of a patient comprising audio of the patient speaking; synthesizing, using the voice sample, audio of a target text read in a voice matching the patient's own voice; presenting the target text to the patient via a computing device; instructing the patient to read the target text aloud in synchronization with the synthesized audio; playing the synthesized audio to the patient; recording the patient's speech output during the synchronization; measuring a synchronicity of the patient's speech output relative to the synthesized audio; determining, based on the synchronicity measure, a complexity for a subsequent target text; and generating the subsequent target text based on the determined complexity. In certain embodiments, a video sample of the patient's face may be obtained and used to synthesize mouth movements of the patient producing the target text, synchronized with the synthesized audio, for display to the patient during the synchronization.

### CROSS-REFERENCE TO RELATED APPLICATIONS

This application claims priority to U.S. Provisional Application No. 63/789,666, filed on Apr. 16, 2025, the entire contents of which are hereby incorporated by reference in their entirety.

### BACKGROUND

Speech and language, while closely related, are distinct domains. Speech refers to the acoustic, phonetic, and articulatory properties of spoken output, including the physical signal produced by the vocal tract. Language refers to the linguistic, syntactic, semantic, and pragmatic properties of the message being communicated, including the cognitive and structural organization of meaning. For individuals with acquired or developmental communication disorders, both speech and language may be impaired. Aphasia, for example, is a condition arising from neurological events (such as stroke, traumatic brain injuries, or degenerative diseases) and is characterized by fragmented and constrained speech outputs that significantly impact quality of life. Individuals with aphasia often experience difficulty forming words, constructing sentences, and maintaining fluency in spontaneous speech, leading to substantial challenges in personal, social, and professional interactions.

Speech entrainment is a well-documented phenomenon in which speakers temporally and prosodically align their speech production to an external auditory model, and has been shown to support fluency in individuals with nonfluent aphasia when a concurrent model is present. Conventional speech entrainment approaches typically employ a non-personalized external voice model (e.g., a clinician's voice or a generic synthesized voice) as the entrainment stimulus. While such approaches have demonstrated therapeutic benefit, they do not leverage the heightened neurological engagement associated with self-referential stimuli. Research in cognitive neuroscience has established that individuals exhibit enhanced attentional orientation and memory encoding for self-referential stimuli, including one's own voice and face. Existing speech entrainment systems do not provide a mechanism for generating a corrected, fluent version of a patient's own voice as the entrainment stimulus, nor do they provide automated, quantitative measurement of the patient's synchronization performance or adaptive adjustment of therapy content complexity based on such measurements.

### SUMMARY

The following presents a simplified summary of one or more aspects of the present disclosure in order to provide a basic understanding of such aspects. This summary is not an extensive overview of all contemplated features of the disclosure, and is intended neither to identify key or critical elements of all aspects of the disclosure nor to delineate the scope of any or all aspects of the disclosure. Its purpose is to present certain concepts of one or more aspects of the disclosure in a simplified form as a prelude to the more detailed description that is presented later.

The present disclosure provides systems and methods for providing mirror speech entrainment (MSE)-based speech therapy to individuals with communication disorders. In certain embodiments, the disclosed systems capture a patient's own voice and, optionally, facial appearance, process those recordings through one or more AI-based synthesis models to generate a corrected, fluent version of the patient's speech in the patient's own voice and likeness, present that synthesized output back to the patient as an entrainment stimulus, and adaptively adjust the complexity of subsequent therapy content based on quantitative measures of the patient's synchronization performance. By making the entrainment stimulus self-referential—derived from the patient's own voice—the disclosed systems may leverage heightened neurological engagement associated with self-referential stimuli to produce greater gains in lexical retrieval and speech motor fluency than conventional, non-personalized entrainment approaches.

In one aspect, the present disclosure provides systems and methods that perform speaker-adaptive neural speech synthesis, in which a speaker encoder extracts a compact speaker identity representation from a patient's own voice sample—including voice samples exhibiting disordered or disfluent speech characteristics—and a neural text-to-speech decoder generates fluent, errorless synthesized speech conditioned on that representation, producing an entrainment stimulus that preserves the speaker-specific acoustic identity of the patient. In further aspects, the disclosed systems perform latency-calibrated synchronicity measurement using signal processing algorithms that detect embedded calibration tones, compute device latency corrections, extract voiced onset times from raw audio waveforms, and produce scalar timing metrics quantifying the temporal alignment between the patient's speech production and the synthesized entrainment stimulus. The disclosed systems further use the quantitative synchronicity metrics as inputs to a decision function that automatically determines the linguistic complexity parameters for subsequent therapy content and generates new target text satisfying those parameters, closing a feedback loop in which measured patient performance data drives automated content generation.

In one aspect, the present disclosure provides a method for providing mirror speech entrainment-based speech therapy. The method may include obtaining a voice sample of a patient comprising audio of the patient speaking; synthesizing, using the voice sample, audio of a target text read in a voice matching the patient's own voice; presenting the target text to the patient via a computing device; instructing the patient to read the target text aloud in synchronization with the synthesized audio; playing the synthesized audio to the patient; recording the patient's speech output during the synchronization; measuring a synchronicity of the patient's speech output relative to the synthesized audio; determining, based on the synchronicity measure, a complexity for a subsequent target text; and generating the subsequent target text based on the determined complexity. In further aspects, the method may include obtaining a video sample of the patient and synthesizing mouth movements of the patient producing the target text synchronized with the synthesized audio. Further aspects include computing devices and non-transitory computer-readable media configured to perform the foregoing operations.

These and other aspects of the disclosure will become more fully understood upon a review of the drawings and the detailed description that follows. Other aspects, features, and embodiments of the present disclosure will become apparent to those skilled in the art upon reviewing the following description of specific, example embodiments of the present disclosure in conjunction with the accompanying figures. While features of the present disclosure may be discussed relative to certain embodiments and figures below, all embodiments of the present disclosure can include one or more of the advantageous features discussed herein. In other words, while one or more embodiments may be discussed as having certain advantageous features, one or more of such features may also be used in accordance with the various other embodiments of the disclosure discussed herein. Similarly, while example embodiments may be discussed below as device, system, or method embodiments, it should be understood that such example embodiments can be implemented in various devices, systems, and methods.

### BRIEF DESCRIPTION OF THE DRAWINGS

FIG. 1 is a block diagram illustrating an environment for mirror speech entrainment (MSE)-based speech therapy according to an embodiment of the present disclosure.

FIG. 2 is a block diagram illustrating a processing pipeline for mirror speech entrainment therapy according to an embodiment of the present disclosure.

FIG. 3 is a flowchart illustrating a method for providing mirror speech entrainment-based speech therapy according to an embodiment of the present disclosure.

FIG. 4 is an illustration of example audio waveforms and synchronicity measurements for assessing speech synchronization performance during a mirror speech entrainment therapy session according to an embodiment of the present disclosure.

FIG. 5 is a block diagram illustrating a computing environment suitable for implementing the systems and methods for mirror speech entrainment therapy disclosed herein.

### DETAILED DESCRIPTION

The following Detailed Description is organized as follows. First, terminology and definitional conventions used throughout this disclosure are introduced. Next, an overview of the system architecture is provided, describing the principal hardware and software components and their functional relationships. The disclosure then describes a series of example operational embodiments, including fully autonomous deployment, clinician-guided deployment, and hybrid deployment modes that may combine elements of both. Thereafter, a step-by-step description of the method flow is provided, including patient onboarding and profile initialization, session initiation and stimulus presentation, real-time synchronicity measurement, adaptive content and difficulty selection, and session logging and reporting. The disclosure then describes the model training and inference processes underlying speaker-adaptive audio synthesis and optional visual synthesis. A description of example implementations and experimental validation follows. The embodiments described in different sections of this disclosure are not mutually exclusive; features described in the context of one embodiment may be combined with features described in the context of other embodiments unless the context clearly dictates otherwise.

The following detailed description is provided to enable any person skilled in the relevant art, including speech-language pathologists, clinical linguists, and AI/ML engineers, to make and use the disclosed subject matter. The described embodiments are intended as non-limiting examples, and the scope of the disclosure is defined by the appended claims. Unless otherwise stated, the term “comprising” and its variants are to be understood in an open-ended sense. All reference numerals appearing herein may correspond to the elements labeled in the accompanying drawings.

The present disclosure describes systems, methods, and computer-readable media for providing mirror speech entrainment (MSE)-based speech therapy to individuals with communication disorders. The systems and methods are described herein primarily in the context of nonfluent aphasia and related conditions arising from neurological events such as stroke, as a non-limiting example. However, the technology described herein may be broadly applicable across a wide range of communication and motor speech disorders, and the scope of the disclosure is not limited to aphasia or neurological etiologies. For example, the described systems and methods may be applied to individuals with motor speech disorders, including apraxia of speech and dysarthria of neurological or non-neurological origin. The technology may be applied to individuals with primary progressive aphasia (PPA) or other neurodegenerative conditions affecting language and speech production, enabling longitudinal therapy that may be adapted to gradual changes in communication ability. The described systems and methods may further be applied to individuals with communication disorders arising from traumatic brain injury (TBI), regardless of aphasia classification. Beyond acquired communication disorders, the technology may be used for accent modification, enabling speakers who wish to modify phonemic or prosodic features of their speech to practice against a personalized synthesized model of their own voice producing target-accent speech. The technology may similarly be used for second-language pronunciation training, in which a speaker's native-language voice characteristics may be preserved in a synthesized model producing target-language phonology with native-like or target-like accuracy, enabling highly personalized second-language acquisition practice. In certain embodiments, the described systems and methods may be applied to any context in which a speaker may benefit from practicing against a personalized, corrected, or otherwise modified version of their own voice and, optionally, their own visual articulatory movements. The description herein, including all examples and figures, is presented in terms of nonfluent aphasia and neurological events as non-limiting illustrative examples only.

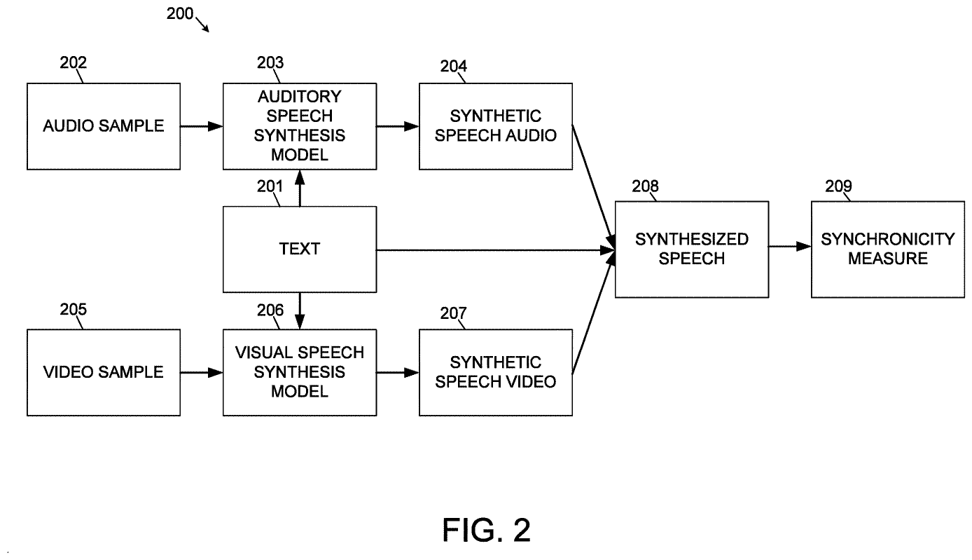

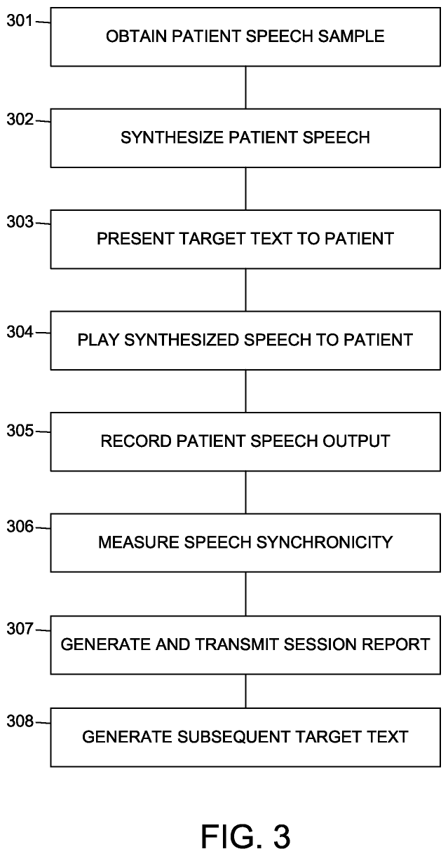

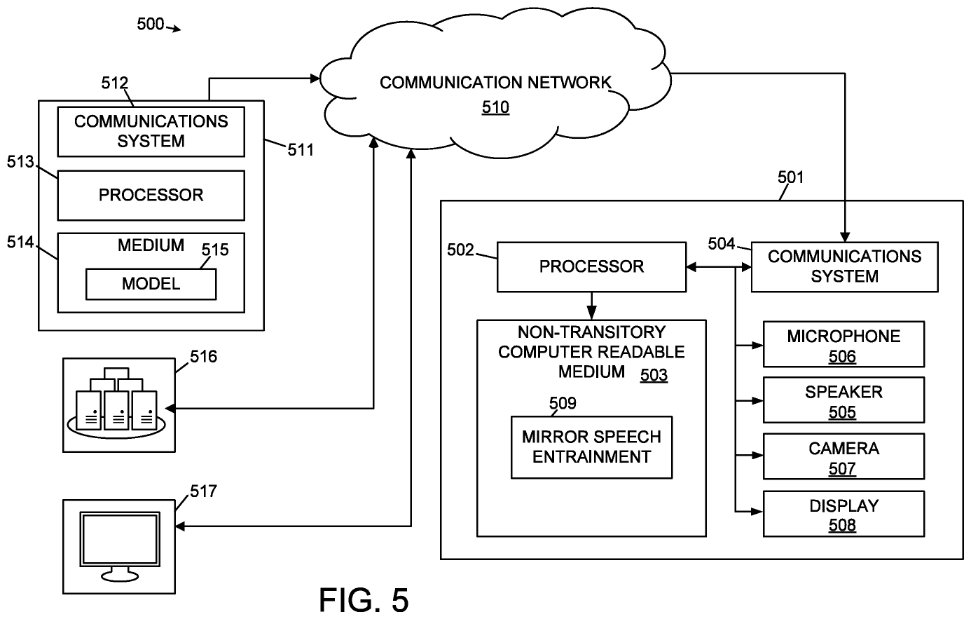

In certain embodiments, the disclosed systems and methods may enable a computing device to capture a patient's own voice and, optionally, facial appearance, process those recordings through one or more AI-based synthesis models to generate a corrected, fluent version of the patient's speech in the patient's own voice and likeness, present that synthesized output back to the patient as an entrainment stimulus, and adaptively adjust the complexity of subsequent therapy content based on quantitative measures of the patient's synchronization performance. The described embodiments may be implemented as stored instructions on one or more computer-readable mediums executed by processors on devices such as user devices, mobile computing devices, desktop computers, or servers accessible via a communication network. While the following description refers to specific figures and components, it will be appreciated by one skilled in the art that the entire pipeline of FIG. 2, the method steps of FIG. 3, and the computer architecture of FIG. 5 may each be implemented fully or in part via stored instructions on one or more non-transitory computer-readable mediums executed by one or more processors, whether those processors are located on a user device, a remote server, or some combination thereof.

The theoretical basis for MSE may be grounded in a sophisticated integration of established frameworks from cognitive neuroscience, neuroplasticity science, and evidence-based aphasia intervention research. These frameworks may include: (1) the Self-Attention Network (SAN), which describes heightened attentional orientation toward self-referential stimuli such as one's own name, voice, or face; (2) the Self-Reference Effect, which describes enhanced memory encoding and neural engagement for information processed in relation to oneself; (3) Speech Entrainment, the well-documented phenomenon in which speakers may temporally and prosodically align their speech production to an external auditory model, supporting fluency in nonfluent aphasia when a concurrent model is present; (4) Script-based Intervention approaches, in which patients may practice personalized, pre-scripted sentences to build automaticity and generalization of functional speech; and (5) Principles of Experience-Dependent Neuroplasticity, which establish that repetitive, specific, salient, and progressively challenging practice may drive cortical reorganization and motor learning in post-stroke rehabilitation. MSE may synthesize these frameworks by making the entrainment stimulus self-referential (the corrected, synthesized version of the patient's own voice and face).

In the field of speaker-adaptive neural speech synthesis, the disclosed system may condition a neural text-to-speech synthesis model on a compact speaker identity representation extracted from a patient's own voice sample—including voice samples exhibiting disordered or disfluent speech characteristics—to generate fluent, errorless synthesized speech that preserves the speaker-specific acoustic identity of the patient. This speaker-adaptive synthesis pipeline, described in detail with respect to the auditory speech synthesis model 203 and block 302 of FIG. 3, may involve computational operations including speaker embedding extraction, decoder-level conditioning on the speaker embedding, and output evaluation against the patient's speaker identity representation. These operations may constitute a sequence of data transformations performed by model architectures operating on data representations to produce synthesized speech that may be simultaneously fluent and speaker-matched to a disordered speaker.

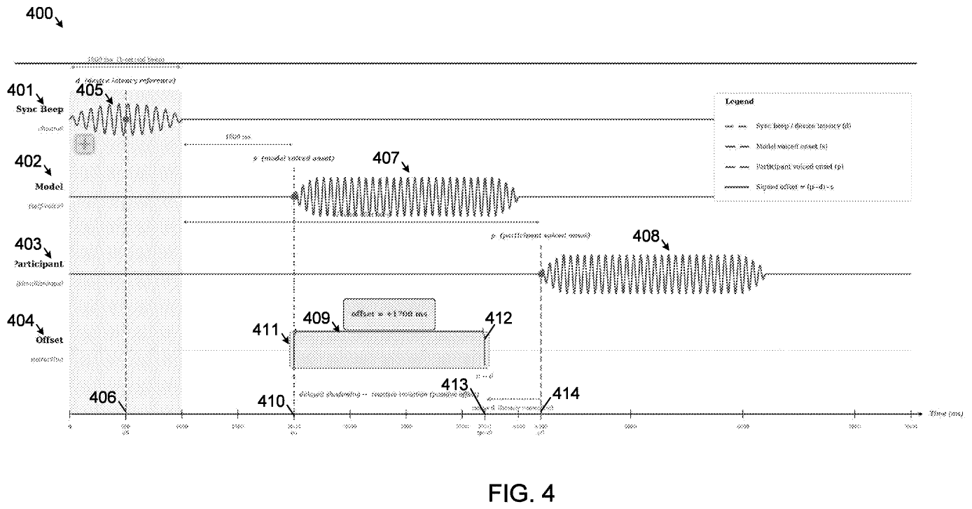

In the field of audio-visual synchronization signal processing, the disclosed system may perform latency-calibrated, signal-level acoustic analysis of a patient's live speech output relative to a synthesized model speech signal to produce a set of scalar timing metrics quantifying the temporal alignment between the patient's speech production and the synthesized entrainment stimulus. This synchronicity measurement pipeline, described in detail with respect to FIG. 4 and block 306 of FIG. 3, may involve signal processing operations including embedded calibration tone detection, device latency computation, voiced onset time extraction from raw audio waveforms, signed offset computation, and statistical aggregation of offset values across multiple repetitions. These operations may produce machine-readable scalar values stored in defined data structures.

In the field of quantitative adaptive content selection in human-computer interaction systems, the disclosed system may use the quantitative synchronicity metrics produced by the signal processing pipeline as inputs to a decision function that may automatically determine the linguistic complexity parameters for subsequent therapy content and generate new target text satisfying those parameters. This adaptive content selection pipeline, described in detail with respect to block 308 of FIG. 3, may close a feedback loop in which measured patient performance data drives automated content generation without requiring human intervention, thereby enabling continuous, real-time adaptation of therapeutic content complexity that may not be achievable through manual clinical assessment.

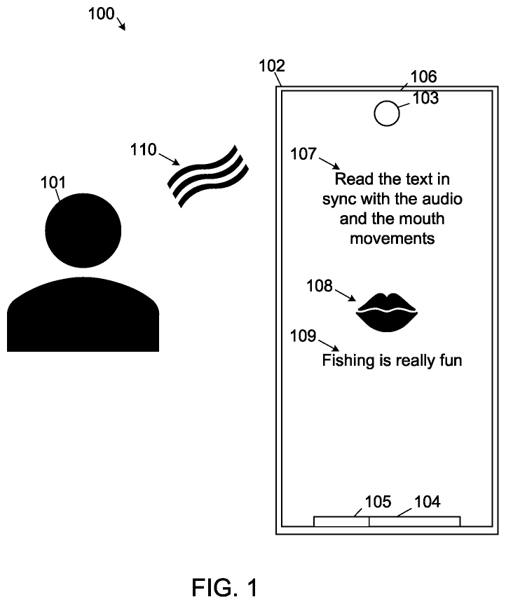

FIG. 1 illustrates an environment 100 for mirror speech entrainment (MSE)-based speech therapy. The environment 100 may depict a patient participating in a session of MSE therapy using a computing device 102. In a broad sense, FIG. 1 may be understood to capture all of the high-level elements of an MSE therapy session: the patient may provide a speech sample to the system, the system may generate and present a personalized entrainment stimulus, and the patient may practice reading target text in synchronization with that stimulus. Each of the elements described herein with respect to FIG. 1 may be supported by the more detailed pipeline of FIG. 2, the method flowchart of FIG. 3, and the computer architecture of FIG. 5.

The computing device 102 may be any suitable computing device, including without limitation a smartphone, tablet, laptop computer, desktop computer, or dedicated therapeutic device. In certain embodiments, the computing device 102 may be a mobile device operated by the patient in a home or clinical setting, enabling the patient to engage in self-directed therapy sessions outside of a formal clinical appointment. In further embodiments, the computing device 102 may be a workstation or tablet operated in a clinical environment under the supervision of a speech-language pathologist (SLP). The computing device 102 may be configured to communicate with one or more remote servers via a communication network, as further described with respect to FIG. 5.

The computing device 102 may be equipped with a microphone 105 for capturing the patient's spoken utterances and a camera 103 for capturing video of the patient's face, and in particular of the patient's mouth and perioral region, during speech production. The microphone 105 and camera 103 may collectively provide the audio and video input through which the patient's voice characteristics and facial movement patterns are sampled, as described further with respect to blocks 301 and 302 of FIG. 3. The computing device 102 may further comprise a display 106 for presenting visual information to the patient during a therapy session and a speaker 104 for delivering audio output to the patient. As illustrated in FIG. 1, the display 106 may present target text 109 for the patient to read, as well as a synthesized mouth depiction 108 comprising a synthesized rendition of the patient's own mouth and perioral region producing the target text. The audio waves 110 depicted in FIG. 1 may represent the synthesized audio played to the patient via the speaker 104, which audio may comprise the patient's own voice—synthesized to be fluent and errorless—reading the target text 109.

During a therapy session, the instruction 107 may be presented by the system to the patient, directing the patient to read the target text 109 aloud in synchronization with the synthesized audio played through the speaker 104 and, where visual synthesis is employed, in synchronization with the synthesized mouth movements displayed on the display 106. This simultaneous presentation of synthesized audio and visual stimuli—each derived from the patient's own voice and appearance—may constitute the core entrainment mechanism of MSE therapy. The patient's brain may be exposed to a corrected, fluent version of its own speech signal, which may leverage the self-attention network and self-reference effect to heighten neurological engagement with the entrainment stimulus.

In certain embodiments, the system may employ speaker-adaptive neural speech synthesis, in which a speaker encoder may extract a compact speaker identity representation from the patient's own voice sample and a neural text-to-speech decoder may generate fluent speech conditioned on that representation, producing an entrainment stimulus that may be both fluent and speaker-matched to the patient.

In certain embodiments, the system may perform latency-calibrated synchronicity measurement, in which defined signal processing algorithms may perform acoustic onset detection on raw audio waveforms, may compute device latency corrections using an embedded calibration tone, and may produce a set of scalar timing metrics—including signed offset, median offset, standard deviation of offsets, mean asynchrony, and phase alignment—that quantify synchronization performance with millisecond-level precision. These metrics may be computed automatically by the system's processor, stored in defined data structures, and used as direct inputs to the adaptive complexity determination algorithm.

As described in further detail with respect to FIG. 2 and FIG. 3, the system may first obtain an audio sample of the patient speaking (via the microphone 105) and, in certain embodiments, a video sample of the patient's face during speech (via the camera 103). These samples may be processed through an auditory speech synthesis model and, optionally, a visual speech synthesis model to generate synthesized speech audio and synthesized speech video, respectively. The synthesized outputs may then be presented back to the patient as an entrainment stimulus during the therapy session, while the patient's live speech output during entrainment may be recorded and analyzed to compute a synchronicity measure. That synchronicity measure may drive the adaptive selection of subsequent target text, as described with respect to blocks 306 through 308 of FIG. 3.

FIG. 2 illustrates a processing pipeline 200 for mirror speech entrainment therapy. The pipeline 200 may depict the sequence of data inputs, processing components, and outputs that together may constitute the MSE system. While FIG. 2 is illustrated as a pipeline, each component therein may be implemented via stored instructions on one or more non-transitory computer-readable mediums executed by processors on devices such as user devices or servers. The processing performed by the pipeline 200 may be distributed across one or more computing devices, including a user device such as computing device 102 of FIG. 1 and one or more remote servers, as described further with respect to FIG. 5.

The input target text 201 may be the text that the patient is directed to read aloud during the therapy session. The target text 201 may comprise sentences, phrases, or other linguistic units that may have been selected based on the patient's current level of performance, selected linguistic targets specified by a clinician, or the patient's personal preferences. As described further with respect to block 303 of FIG. 3, the selection of the target text 201 may be dynamically adjusted across sessions based on the patient's synchronicity measure, accumulated synchronicity data from prior sessions, and complexity parameters determined by the system. The target text 201 may be presented to the patient on the display 106 of the computing device 102.

The audio sample 202 may be a recording of the patient's own voice captured by the microphone 105 of the computing device 102 while the patient reads or speaks a set of sample utterances. The audio sample 202 may comprise a brief sample of the patient's natural, post-stroke or otherwise disordered speech. This sample may be used to derive an acoustic representation of the patient's voice characteristics—including fundamental frequency, formant structure, prosodic patterns, and speaker-specific timbre—sufficient to enable a voice cloning or voice synthesis model to generate new speech in a voice that matches the patient's own voice. The audio sample 202 need not be errorless; rather, it may be an authentic sample of the patient's current speech production that is used as a basis for the auditory speech synthesis model 203.

The auditory speech synthesis model 203 may be a machine learning model, such as a voice cloning model or a neural text-to-speech model conditioned on the patient's voice characteristics, that takes as input the audio sample 202 and the target text 201 and generates as output the synthetic speech audio 204. The auditory speech synthesis model 203 may be implemented using any suitable neural speech synthesis architecture, including without limitation a neural text-to-speech (TTS) model conditioned on a speaker embedding derived from the audio sample 202, a voice conversion model that may transform a reference TTS voice into the patient's voice, or a voice cloning model that may encode the patient's voice characteristics and decode synthesized speech in that voice. The auditory speech synthesis model 203 may be accessed locally on the user device or via a remote server through a network API call. As described with respect to block 302 of FIG. 3, the auditory speech synthesis model 203 may generate fluent, errorless synthesized speech in the patient's own voice reading the target text 201.

The auditory speech synthesis model 203 may implement a pipeline for speaker-adaptive synthesis. In certain embodiments, the pipeline may comprise three principal stages. First, a speaker encoder network may process the audio sample 202 to extract a fixed-dimensional speaker embedding vector—a compact numerical representation of the patient's speaker identity that may encode voice characteristics including fundamental frequency range, formant structure, spectral envelope shape, and speaker-specific timbre. The speaker encoder may be implemented as a recurrent neural network, a convolutional neural network, or a transformer-based encoder trained on a speaker verification or speaker identification objective, such that the resulting embedding vector may occupy a position in a learned speaker embedding space where proximity corresponds to speaker similarity. Second, a neural text-to-speech decoder—which may be implemented as an autoregressive sequence-to-sequence model, a non-autoregressive parallel decoder, or a diffusion-based generative model—may generate a mel-spectrogram or other intermediate acoustic representation of the target text 201, conditioned at the decoder level on the patient's speaker embedding vector. This conditioning may cause the decoder to generate acoustic features that reflect the phonetic content of the target text while preserving the speaker-specific acoustic characteristics encoded in the embedding. A vocoder network may then convert the mel-spectrogram into a time-domain audio waveform constituting the synthetic speech audio 204. Third, the system may evaluate the fidelity of the synthesized output by computing a cosine similarity metric between the speaker embedding extracted from the synthetic speech audio 204 and the speaker embedding extracted from the patient's original audio sample 202, providing a quantitative, machine-computable measure of speaker identity preservation.

The synthetic speech audio 204 may be the output of the auditory speech synthesis model 203: a synthesized audio rendering of the patient's own voice reading the target text 201 in a fluent, errorless manner. The synthetic speech audio 204 may be subsequently combined with any visual synthesis output (described below) to produce the synthesized speech 208, which may be the complete entrainment stimulus delivered to the patient. In certain embodiments, the synthetic speech audio 204 may be produced in one or more entrainment modes, including without limitation: a conversational mode that may produce synthesized speech with naturalistic prosody and variable inter-word timing characteristic of spontaneous speech, or a melodic mode that may produce synthesized speech with exaggerated pitch contours, regular isochronous rhythmic timing, and amplified lexical stress contrasts. The conversational and melodic modes are described in further detail with respect to block 302 of FIG. 3. The selection between these entrainment modes is also described further with respect to block 302 of FIG. 3.

In embodiments that also capture a video sample of the patient, the pipeline 200 may further comprise a video sample 205, a visual speech synthesis model 206, and a synthetic speech video 207. The video sample 205 may be a recording of the patient's face, captured by the camera 103, while the patient produces the sample speech. The video sample 205 may provide visual information about the patient's mouth shape, lip movements, jaw movements, and perioral facial region during speech production. The visual speech synthesis model 206 may be a machine learning model—such as a neural talking head model, a face reenactment model, or a deepfake-style synthesis model—that takes as input the video sample 205 and the synthetic speech audio 204, and generates as output the synthetic speech video 207. The visual speech synthesis model 206 may synthesize the patient's mouth and perioral region producing the target text 201 with correct articulation, synchronized to the timing of the synthetic speech audio 204. The synthetic speech video 207 may depict the patient's own mouth and perioral region—focused on the mouth and the area immediately surrounding it—producing fluent, errorless speech movements synchronized with the synthetic speech audio 204.

In various embodiments, the generated or synthesized video comprises a view focused on the oral and perioral region of a subject, such that nonessential facial regions are excluded while preserving anatomically relevant structures associated with speech articulation. The framed region may include one or more anatomical landmarks selected from the group consisting of the subnasal region, the nasolabial region, the vermilion border of the lips, the oral commissures, the perioral musculature, the mentolabial region, and the mental protuberance.

In one example embodiment, the framed region extends superiorly from a subnasale landmark located immediately inferior to the nasal septum and inferior nasal ala, and extends inferiorly to a menton or chin region, thereby encompassing the upper lip (labium superius), lower lip (labium inferius), oral aperture, vermilion zones, and surrounding perioral musculature, including portions of the orbicularis oris, levator labii superioris, depressor labii inferioris, and related facial muscles involved in speech articulation.

In another example embodiment, the framed region is limited to the moving components of the oral articulatory system, extending superiorly to an upper vermilion border of the labium superius and inferiorly to a region immediately below the lower vermilion border of the labium inferius, optionally including a portion of the sublabial or mentolabial region sufficient to capture lower-lip displacement during phonation. In this embodiment, non-articulatory structures such as the nasal base and chin may be excluded from the generated video.

In a further embodiment, the framed region includes the oral commissures bilaterally and adjacent perioral skin, while excluding the nose, cheeks beyond a predetermined lateral distance from the oral commissures, and non-moving portions of the mandible. Such selective framing emphasizes dynamic deformation of soft tissue associated with speech, including lip protrusion, retraction, rounding, closure, and lateral spreading.

In some embodiments, the spatial boundaries of the framed region are defined anatomically rather than geometrically, such that the boundaries correspond to clinically recognized facial subunits. The framed region may correspond to some or all of the perioral subunit, optionally including the upper cutaneous lip, lower cutaneous lip, labial tubercle, and mentolabial fold, while excluding supra-labial, infra-mental, or lateral buccal regions.

In some implementations, the framing region is dynamically adjusted during generation to include only those anatomical regions exhibiting motion above a threshold associated with speech articulation, such that static surrounding anatomy is cropped or suppressed. In such cases, the framed region may vary over time while remaining constrained to recognized oral and perioral anatomical boundaries.

For purposes of this disclosure, “mouth region,” “oral region,” and “perioral region” refer to anatomical areas associated with speech-related movement of the lips and surrounding soft tissue, and are not limited to any particular pixel dimensions, aspect ratio, or absolute facial scale.

The synthesized speech 208 may represent the complete, combined entrainment stimulus: the synthetic speech audio 204 (which may be played via the speaker 104) and, where applicable, the synthetic speech video 207 (which may be displayed on the display 106 as the synthesized mouth 108 of FIG. 1). The synthesized speech 208 may be presented to the patient during the therapy session while the patient simultaneously reads the target text 201 displayed on the display 106 and attempts to synchronize their own speech output with the synthesized stimulus.

The synchronicity measure 209 may be a quantitative value computed from the patient's recorded live speech output during the entrainment exercise, measuring the degree to which the patient's speech onset timing aligns with the onset timing of the synthesized speech 208. The synchronicity measure 209 is described in further detail with respect to FIG. 4 and block 306 of FIG. 3. The synchronicity measure 209 may drive the adaptive selection of subsequent target text, as described with respect to block 308 of FIG. 3.

In one set of embodiments, the speech therapy system may operate in a fully autonomous mode in which no contemporaneous clinician involvement is required during a therapy session. In autonomous mode, therapy parameters (including target phoneme sets, practice text corpus, session duration, initial difficulty level, and acceptable synchronicity thresholds) may be initialized during an onboarding process (described further below) and stored in a patient profile data structure accessible to the system. During each session, the system may independently present entrainment stimuli, capture patient speech attempts, compute synchronicity metrics, adapt content difficulty, and log session results, all without requiring real-time input from a licensed clinician. Autonomous mode may be suited to patients who have completed initial clinician-guided onboarding and are engaged in maintenance or generalization phases of therapy, or to patients in geographic or scheduling contexts in which regular clinician availability may be limited.

In certain embodiments, the method may comprise operating the system in an autonomous mode in which the computing device independently presents entrainment stimuli, captures patient speech attempts, computes synchronicity metrics, adapts content difficulty, and logs session results without requiring contemporaneous clinician involvement during the therapy session. In certain embodiments, the method may comprise detecting, in autonomous mode, a pattern of sustained performance decline comprising synchronicity scores falling below a defined threshold across a configurable number of consecutive sessions, and in response, generating an alert notification and transmitting the alert notification to an associated clinician account, and suspending autonomous difficulty progression pending clinician review.

In certain embodiments, the method may comprise operating the system in an autonomous mode in which the computing device independently presents entrainment stimuli, captures patient speech attempts, computes synchronicity metrics, adapts content difficulty, and logs session results without requiring contemporaneous clinician involvement during the therapy session. In certain embodiments, the method may comprise detecting, in autonomous mode, a pattern of sustained performance decline comprising synchronicity scores falling below a defined threshold across a configurable number of consecutive sessions, and in response, generating an alert notification and transmitting the alert notification to an associated clinician account, and suspending autonomous difficulty progression pending clinician review.

In certain embodiments, autonomous mode may include passive safety and escalation logic. For example, if a pattern of sustained performance decline is detected by the system (such as synchronicity scores falling below a defined threshold across a configurable number of consecutive sessions), an alert flag may be generated in the patient's session log and, in embodiments with clinician-oversight connectivity, a notification may be transmitted to the associated clinician account. The system may also automatically suspend autonomous progression and maintain the current difficulty level pending clinician review, rather than continuing to escalate content difficulty in a manner that may be inappropriate for a patient experiencing a regression.

In another set of embodiments, the system may operate in a clinician-guided mode in which a licensed SLP may actively monitor and direct therapy in real time or near-real time through a clinician interface. The clinician interface may comprise a web-based dashboard or a dedicated software application accessible via a networked device, and may display real-time session data including a live or buffered feed of the patient's speech attempts, computed synchronicity scores for each attempt, entrainment stimulus playback, and patient performance trend graphs.

In clinician-guided mode, override authority over adaptive content selection decisions made by the system may be retained by the SLP. For example, specific practice texts may be manually selected by the SLP, difficulty parameters may be adjusted mid-session, or the stimulus-presentation cycle may be paused and restarted. The clinician interface may also allow the SLP to annotate individual patient attempts (flagging specific attempts as requiring additional review, marking observed articulatory patterns, or appending text notes), which annotations may then be stored in the session log alongside the system-generated quantitative metrics.

Clinician-guided mode may be particularly suited to the early phases of therapy, to patients with more severe or complex presentations, or to evaluation sessions in which the SLP may wish to conduct a structured assessment of the patient's baseline or progress using the system's quantitative measurement capabilities as a clinical instrument.

In yet another set of embodiments, the system may support a hybrid deployment mode that may combine elements of autonomous and clinician-guided operation. In hybrid mode, a majority of sessions may be conducted autonomously by the patient—interacting with the system through the patient-facing interface without a clinician present—while session logs, trend data, and performance reports may be periodically reviewed by the clinician through the clinician portal, and therapy parameters may be adjusted between sessions based on that review.

In certain hybrid embodiments, the system may be configured so that parameter adjustments made by the clinician through the portal take effect at the beginning of the patient's next scheduled session, without requiring the patient and clinician to interact in real time. For example, after reviewing a week's worth of session logs, the target text corpus may be updated by the SLP, the synchronicity threshold for difficulty advancement may be modified, or a new phoneme category may be added to the practice set—and these updates may be pushed to the patient profile data structure so that they govern the next autonomous session.

Hybrid deployment mode may provide a balance between the efficiency and access advantages of autonomous operation and the clinical oversight advantages of periodic SLP review, and may be well suited to telehealth deployment contexts.

In certain embodiments, a patient profile data structure may be maintained by the system for each patient enrolled in the system. The patient profile may include, without limitation: patient identifier information; an initial speaker model derived from recorded patient speech samples; a current difficulty level parameter; a target phoneme or phoneme-class set defining the speech targets for the current therapy phase; an active practice text corpus or corpus selection parameters; synchronicity threshold parameters defining the performance criteria for difficulty advancement and regression; session frequency and duration parameters; clinician association data linking the patient profile to one or more supervising SLP accounts; and cumulative session history data.

Patient profile initialization may be performed during an onboarding process that may include one or more of the following steps. First, demographic and clinical background information may be provided by the patient or a clinician through the patient-facing interface or clinician portal. Second, the patient may be prompted by the system to produce a series of calibration speech samples—for example, reading aloud a standard passage—from which acoustic and prosodic features may be extracted by the speaker-adaptive synthesis model for use in generating personalized entrainment stimuli. Third, initial therapy parameters including starting difficulty level and initial target phoneme set may be set by the system or clinician, either through clinician input or through automated assessment of the patient's baseline synchronicity performance on a standardized stimulus set. Fourth, the patient profile data structure may be stored in association with the patient's account, and may be updated on a session-by-session basis as therapy progresses.

In certain embodiments, the speaker model component of the patient profile may be periodically updated as additional patient speech data is collected across sessions, so that the entrainment stimuli generated by the system may remain acoustically calibrated to the patient's evolving voice characteristics over the course of therapy.

In certain embodiments, the system may include scheduling functionality that supports configuring a recurring or variable session schedule for each patient. Scheduling parameters may include session frequency (e.g., daily, on specified days of the week, or on a clinician-defined schedule), session duration, and preferred session time windows. Scheduling data may be stored as part of the patient profile data structure and may be modifiable by the patient, by an associated clinician, or by both, depending on account configuration.

In embodiments with notification functionality, session reminder notifications may be generated and delivered by the system to the patient through one or more communication channels, which may include push notifications delivered through a patient-facing application, electronic mail notifications, or short message service (SMS) notifications. Reminder notifications may be configurable as to timing—for example, a first reminder may be generated a designated interval before the scheduled session time, and a second reminder may be generated if the session has not been initiated within a defined window after the scheduled time.

In certain embodiments, the system may also support spaced practice scheduling in which session intervals may be dynamically adjusted based on the patient's demonstrated retention and performance across sessions, consistent with principles of spaced repetition known in the art of learning science and motor skill acquisition. For example, a patient demonstrating consistent synchronicity scores above a defined threshold across multiple consecutive sessions may have their next scheduled session interval extended, while a patient demonstrating variable performance may maintain a higher session frequency.

In certain embodiments, the method may comprise dynamically adjusting a session interval between successive therapy sessions based on the patient's synchronicity measures across a plurality of prior sessions, wherein the session interval is extended in response to the patient demonstrating synchronicity scores above a defined retention threshold across a defined number of consecutive sessions, and the session interval is shortened or maintained in response to the patient demonstrating variable synchronicity scores.

In certain embodiments, the method may comprise dynamically adjusting a session interval between successive therapy sessions based on the patient's synchronicity measures across a plurality of prior sessions, wherein the session interval is extended in response to the patient demonstrating synchronicity scores above a defined retention threshold across a defined number of consecutive sessions, and the session interval is shortened or maintained in response to the patient demonstrating variable synchronicity scores.

In certain embodiments, a comprehensive session log may be maintained by the system for each completed therapy session. The session log may include, without limitation: a session identifier and timestamp; patient identifier; session duration; a record of each practice text presented during the session; the synthesized entrainment stimulus audio data or a reference identifier for each stimulus; a recording of the patient's speech attempt corresponding to each stimulus; the computed synchronicity score and associated latency offset for each attempt; the difficulty level active at the start and end of the session; any difficulty level transitions that occurred during the session and the criteria that triggered each transition; and any flags or alerts generated during the session.

Session logs may be stored in a database associated with the patient's account and may be made accessible to associated clinician accounts through the clinician portal. In certain embodiments, session log data may be aggregated by the system across multiple sessions to compute trend metrics, which may include, without limitation: mean synchronicity score per session over a defined time window; rate of difficulty advancement or regression over a defined time window; session completion rate (ratio of completed sessions to scheduled sessions); variability of synchronicity scores across attempts within a session; and phoneme-class-specific synchronicity metrics where the system maintains phoneme-level scoring.

In certain embodiments, structured progress reports may be generated by the system on a configurable schedule—for example, weekly or monthly—or upon clinician request. Progress reports may present trend metrics in human-readable format, including graphical representations of synchronicity score trajectories and difficulty progression over time, and may be accessible through the clinician portal or exportable in a standard document format. Such reports may be used by clinicians to inform therapy parameter adjustments, to document patient progress for clinical recordkeeping purposes, or to support insurance reimbursement documentation.

In certain embodiments, security and privacy measures appropriate for deployment in a healthcare context may be implemented by the system. Patient data—including speech recordings, session logs, and patient profile data—may be transmitted between client devices and server-side components over encrypted communication channels, for example using transport layer security (TLS) or equivalent protocols. Patient data stored on server-side components may be encrypted at rest using industry-standard encryption methods.

In certain embodiments, access to patient data through the clinician portal may be controlled through authentication mechanisms, which may include password-based authentication, multi-factor authentication, or single sign-on integration with institutional identity management systems. Access control logic may be configured to enforce that each clinician account has access only to data associated with patients linked to that clinician's account, and that patient accounts have access only to their own data.

In certain embodiments, the system may be configured to operate in compliance with applicable healthcare data privacy regulations, including, for example, the Health Insurance Portability and Accountability Act (HIPAA) in the United States. Specific compliance features may include audit logging of access to patient records, configurable data retention policies, and de-identification functionality for research or analytics use cases in which individual patient data may be aggregated.

FIG. 3 is a flowchart illustrating a method 300 for providing mirror speech entrainment-based speech therapy. The blocks of FIG. 3 are described below in sequence. Because the method blocks of FIG. 3 are stated at a high level of generality, each block may be associated herein with the full range of operations, options, alternatives, and implementation variations covered by the corresponding claim language. The method of FIG. 3 may be implemented in its entirety as stored instructions on one or more non-transitory computer-readable mediums executed by one or more processors on a user device, a server, or any combination thereof, as further described with respect to FIG. 5.

In block 301, a voice sample of the patient comprising audio of the patient speaking may be obtained by the system. The voice sample may be captured by the microphone 105 of the computing device 102. In certain embodiments, a video sample of the patient comprising a recording of the patient's face while speaking may also be obtained by the system, captured by the camera 103 of the computing device 102. In embodiments where only an audio sample is obtained, the system may operate in an auditory-only MSE mode. In embodiments where both audio and video samples are obtained, the system may operate in an audiovisual MSE mode. Both modes fall within the scope of block 301. The patient need not produce error-free speech during the sampling phase; the system may be configured to extract voice characteristics and facial appearance features from the patient's current, potentially disordered speech production. In the auditory-only mode, the audio sample 202 may provide the basis for voice cloning or voice synthesis. In the audiovisual mode, the audio sample 202 and the video sample 205 may together provide the basis for both auditory and visual synthesis. In certain embodiments, obtaining the voice sample may comprise prompting the patient to read a set of seed sentences aloud while the microphone 105 records. The seed sentences may be selected to cover a phonetically diverse set of sounds to maximize the information captured about the patient's voice characteristics.

In certain embodiments, the voice sample obtained in block 301 may comprise audio of the patient speaking with disordered or disfluent speech characteristics, including speech exhibiting one or more of reduced speech rate, phonemic paraphasias, phonetic distortions, articulatory groping, false starts, or agrammatic sentence structures. The system may be configured to extract speaker identity characteristics from such disordered speech samples and to generate fluent, errorless synthesized speech that preserves the speaker-specific acoustic identity of the patient notwithstanding the presence of speech errors or disfluencies in the original voice sample. In certain embodiments, the voice sample may comprise a minimum duration of recorded speech, such as at least thirty seconds or at least sixty seconds of phonetically diverse speech, sufficient to enable extraction of a speaker embedding vector that captures the patient's fundamental frequency range, formant structure, spectral envelope shape, and speaker-specific timbre.

In certain embodiments, the voice sample obtained in block 301 may comprise audio of the patient speaking with disordered or disfluent speech characteristics, including speech exhibiting one or more of reduced speech rate, phonemic paraphasias, phonetic distortions, articulatory groping, false starts, or agrammatic sentence structures. The system may be configured to extract speaker identity characteristics from such disordered speech samples and to generate fluent, errorless synthesized speech that preserves the speaker-specific acoustic identity of the patient notwithstanding the presence of speech errors or disfluencies in the original voice sample. In certain embodiments, the voice sample may comprise a minimum duration of recorded speech, such as at least thirty seconds or at least sixty seconds of phonetically diverse speech, sufficient to enable extraction of a speaker embedding vector that captures the patient's fundamental frequency range, formant structure, spectral envelope shape, and speaker-specific timbre.

In certain embodiments, the quality and sufficiency of the obtained voice sample may be evaluated by the system before proceeding to synthesis. This evaluation may include assessing the duration of the sample (ensuring that a minimum amount of phonetically diverse speech has been captured), assessing signal-to-noise ratio or recording clarity, and assessing the intelligibility or phonetic diversity of the captured utterances. If the captured sample does not satisfy minimum quality or duration thresholds, the patient may be prompted by the system to provide additional sample recordings before synthesis proceeds. For example, a prompt may be displayed by the system indicating that additional speech samples are needed and directing the patient to repeat the seed sentence set or to produce additional utterances. In some embodiments, voice sample data may be iteratively accumulated by the system across multiple sessions, refining the voice embedding and improving synthesis fidelity as additional data is collected over the course of therapy. This iterative sample accumulation may be particularly beneficial for patients with severely impaired speech production, for whom a single brief recording may not provide sufficient acoustic information for high-fidelity voice cloning; in such cases, a preliminary synthesized voice may be generated by the system that is progressively refined as more sample data becomes available.

In certain embodiments, the voice sample may additionally comprise an audiovisual recording—both an audio recording of the patient's speech and a simultaneous video recording of the patient's face and mouth region, captured by the camera 103. The audiovisual recording may provide both the acoustic voice characteristics needed for auditory synthesis and the visual articulatory features—including lip shape, jaw position, and perioral movement patterns—needed for visual speech synthesis. In some examples, the audio-only recording may be sufficient for the auditory-only MSE mode, while both audio and video recordings together may support the audiovisual MSE mode. In either mode, the sampling phase may be structured as a guided prompting session in which the patient may be directed to read a set of seed sentences, with the audio (and optionally video) recording captured by the respective sensors of the computing device 102.

In certain embodiments, the patient speech sample may be captured via a microphone integrated into the patient's computing device, which may include a smartphone, tablet, laptop, or dedicated speech therapy terminal. In other embodiments, an external microphone may be used, including a directional or noise-canceling microphone to improve signal quality in environments with ambient noise. The captured audio may be preprocessed—for example, through noise reduction, normalization, or voice activity detection algorithms—before being passed to downstream processing components. In certain embodiments, a push-to-talk interface element may be provided through which the patient may initiate recording by pressing or holding a designated control, thereby reducing the likelihood of capturing non-speech audio at the beginning and end of each attempt. Alternative embodiments may use automatic voice activity detection to initiate and terminate capture without requiring manual patient input.

In block 302, audio of a target text read in a voice matching the patient's own voice may be synthesized by the system using the voice sample. This synthesis may be performed by the auditory speech synthesis model 203, which may take as input the patient's voice sample (audio sample 202) and the target text 201 and may output the synthetic speech audio 204: a fluent, errorless rendering of the target text in the patient's own voice. In certain embodiments, block 302 may further comprise synthesizing, using the video sample, mouth movements of the patient producing the target text, synchronized with the synthesized audio. This visual synthesis may be performed by the visual speech synthesis model 206, taking as input the video sample 205 and the synthetic speech audio 204 and outputting the synthetic speech video 207, which may depict the patient's own mouth and perioral region producing the target text with correct articulation. The synthesized depiction may be focused specifically on the patient's mouth and the perioral region (the area of the face immediately surrounding the mouth) rather than depicting the patient's full face, enabling the patient to attend closely to articulatory cues. The output of block 302 may be the synthesized speech 208.

In certain embodiments, synthesizing the audio of the target text in block 302 may comprise extracting, from the voice sample, a speaker embedding vector using a speaker encoder neural network, the speaker embedding vector being a fixed-dimensional numerical representation of the patient's speaker identity in a learned embedding space. The speaker embedding vector may then be provided as a conditioning input to a neural text-to-speech decoder, which may generate a mel-spectrogram or other intermediate acoustic representation of the target text conditioned on the speaker embedding vector. A vocoder network may convert the intermediate acoustic representation into a time-domain audio waveform constituting the synthesized audio. In certain embodiments, the system may compute a speaker similarity metric between a speaker embedding extracted from the synthesized audio and the speaker embedding extracted from the patient's original voice sample, and may accept or reject the synthesized audio based on whether the speaker similarity metric exceeds a defined speaker fidelity threshold. The speaker similarity metric may comprise a cosine similarity computed between the two speaker embedding vectors.

In certain embodiments, synthesizing the audio of the target text in block 302 may comprise extracting, from the voice sample, a speaker embedding vector using a speaker encoder neural network, the speaker embedding vector being a fixed-dimensional numerical representation of the patient's speaker identity in a learned embedding space. The speaker embedding vector may then be provided as a conditioning input to a neural text-to-speech decoder, which may generate a mel-spectrogram or other intermediate acoustic representation of the target text conditioned on the speaker embedding vector. A vocoder network may convert the intermediate acoustic representation into a time-domain audio waveform constituting the synthesized audio. In certain embodiments, the system may compute a speaker similarity metric between a speaker embedding extracted from the synthesized audio and the speaker embedding extracted from the patient's original voice sample, and may accept or reject the synthesized audio based on whether the speaker similarity metric exceeds a defined speaker fidelity threshold. The speaker similarity metric may comprise a cosine similarity computed between the two speaker embedding vectors.

Block 302 may encompass all modes of synthesis supported by the system. In the conversational mode, the auditory speech synthesis model 203 may generate synthesized speech with naturalistic prosody, variable inter-word timing, and natural stress patterns characteristic of spontaneous conversational speech, such that the patient may practice with a stimulus that reflects the prosodic demands of real-world spoken communication. In the melodic mode, the model may generate synthesized speech with exaggerated pitch contours (amplified pitch excursion on stressed syllables), isochronous rhythmic timing (equal inter-onset intervals across syllables), and heightened lexical stress contrasts, analogous to the prosodic scaffolding approach employed in melodic intonation therapy. For example, for the utterance “Hello, I like fishing,” the conversational mode may produce natural variable inter-onset intervals with moderate stress on “fish,” while the melodic mode may produce equal isochronous inter-onset intervals across all six syllables (hel-lo-I-like-fish-ing), primary nuclear stress on “FISH” with an exaggerated pitch contour, and reduced prominence on function words (“I,” “-ing”). The mode may be selected either by a clinician through a clinician interface or automatically by the system based on the patient's severity of aphasia and their response to prior sessions.

Block 302 may further encompass the application of individualized timing calibration, which may be implemented to ensure precise alignment between the stimulus onset and the patient's production onset before therapy trials begin. Timing parameters may be calibrated by the system by measuring device latency (as described with respect to element 401 of FIG. 4) and adjusting model output onset timing accordingly.

The synthesis operations performed in block 302—including voice cloning or voice embedding extraction and neural text-to-speech inference, as well as visual speech synthesis using a talking-head or face reenactment model—may be computationally intensive and may in certain embodiments be offloaded to one or more remote servers via the communication network 510. In a typical commercial deployment, the patient's audio sample (and, in the audiovisual mode, the video sample) may be transmitted by the user device 501 to a remote synthesis server, which may perform model inference to generate the synthetic speech audio 204 and, where applicable, the synthetic speech video 207, and may return the synthesized outputs to the user device 501 for storage and subsequent presentation during therapy. In certain embodiments, synthesis may be performed as a preprocessing step prior to the active entrainment portion of the therapy session. For example, synthesis may be performed during an introductory or setup portion of the session—such as during an initial listening phase in which the patient listens to seed stimuli—such that the synthesized stimuli for the entrainment phase may be ready before active entrainment trials begin. In other embodiments, synthesis may be performed at the conclusion of a prior therapy session, so that the synthesized stimuli for the next session may be prepared and stored in advance and may be immediately available at the start of the subsequent session. This pre-session synthesis approach may reduce or eliminate real-time synthesis delay during the active entrainment phase and may be a preferred configuration for ensuring smooth stimulus delivery in consumer device deployments. In further embodiments, synthesis may be performed partially on-device and partially on a remote server, such as where a lightweight speaker embedding extraction step may be performed locally and the full neural text-to-speech inference step may be offloaded to the server.

Regarding the melodic mode synthesis, synthesized speech with isochronous inter-onset intervals across syllables and modified pitch and amplitude characteristics may be generated by the auditory speech synthesis model 203 to produce the exaggerated prosodic structure characteristic of melodic entrainment. The isochronous timing may refer specifically to equal inter-onset intervals between successive syllable onsets—that is, the temporal spacing between the start of each syllable may be held constant across all syllables in the utterance, producing a metronomic rhythmic structure. This isochronous syllabic timing may be implemented, for example, through duration constraints applied during neural TTS synthesis, such as forced-alignment with fixed syllable duration targets, SSML duration markup specifying equal inter-syllable onset intervals, or post-processing rhythm normalization of the synthesized waveform to align syllable onsets to a specified tempo grid. Separately from and in addition to isochronous inter-onset timing, amplified pitch excursion on stressed syllables and heightened amplitude contrast between stressed and unstressed syllables may be applied in the melodic mode. Nuclear stress (the most prominent accent in the utterance) may be placed on the rightmost content word according to the Nuclear Stress Rule (e.g., “FISHing” in the utterance “Hello, I like fishing”), with a large, exaggerated pitch excursion at that syllable. Word-level primary stress may also be amplified (e.g., the second syllable of “heLLO” may receive greater pitch height and amplitude than in conversational speech). Unstressed syllables, including function words (“I”) and unstressed affixes (“-ing”), may be reduced in amplitude relative to stressed syllables, heightening the contrast between stressed and unstressed positions. This amplification of pitch and amplitude on stressed syllables may be achieved through pitch manipulation and amplitude scaling of those syllable regions within the waveform, and may be independent of the isochronous timing structure: in the melodic mode, all syllables may retain equal inter-onset intervals regardless of stress weight, while pitch and amplitude may be varied across syllables to convey the stress hierarchy. This distinction (equal timing with variable pitch and amplitude) may be understood as a key aspect of the melodic mode design. In contrast, in the conversational mode, inter-onset intervals may vary naturally with speech rate and stress patterns, and pitch and amplitude may reflect natural prosodic contours without exaggeration, modeling naturalistic spontaneous speech prosody.

The mode (conversational or melodic) may be selected by a clinician through the clinician interface, based on the clinician's assessment of the patient's severity of aphasia and responsiveness to prior therapy. In certain embodiments, patient-specific input data—such as aphasia severity ratings, communication profile, or formal assessment scores—may be provided by the clinician through the clinician interface at the outset of therapy, and this clinician-provided input may be used by the system to initialize the mode, complexity level, and other session parameters appropriate for that patient. In further embodiments, a mode may be suggested by the system based on accumulated session performance data, which the clinician may then confirm or override through the clinician interface.

In certain embodiments, the synthesis model used to generate the corrected audio entrainment stimulus may be a neural text-to-speech (TTS) model that has been fine-tuned or conditioned on acoustic features extracted from the patient's own speech samples, so that the generated audio may be acoustically similar to the patient's voice in terms of pitch, timbre, and prosodic patterns. The practice text input to the synthesis model may be selected from a pre-defined corpus of practice texts associated with the patient's current therapy phase, or may be dynamically generated to target specific phonemes or phoneme sequences identified as areas for improvement based on prior session performance. In certain embodiments, a corrective transformation may additionally be applied by the synthesis model to the patient's acoustic features so that the generated audio may reflect accurate phonemic production—producing a model of what the patient's voice would sound like producing the target text correctly—rather than simply reproducing the patient's characteristic errors. The corrective transformation may be parameterized by acoustic targets derived from normative speech data or from clinician-specified targets.

In certain embodiments, a visual entrainment stimulus comprising a synthesized video of a facial avatar or photorealistic face rendering producing the target speech utterance with synchronized lip and jaw movements may additionally be generated by the system. The visual stimulus may be generated by a visual synthesis model that maps the audio stimulus output to corresponding facial animation parameters, producing a visual stimulus that may be temporally synchronized with the audio stimulus. In certain embodiments, the visual stimulus may be rendered as a realistic face resembling the patient's own appearance, which may be generated from one or more facial photographs provided during onboarding. In other embodiments, a generic avatar face may be used. The audio and visual stimuli may be presented simultaneously through the patient-facing interface, allowing the patient to observe lip and articulatory movement cues synchronized with the audio model, which may provide additional perceptual information supporting speech imitation beyond what the audio stimulus alone may convey.

In block 303, the target text 201 may be presented by the system to the patient via the computing device 102. The target text 201 may be displayed on the display 106 (corresponding to target text 109 as depicted in FIG. 1) while the patient may be simultaneously directed to read it aloud and synchronize their speech with the synthesized audio and, in the audiovisual mode, with the synthesized mouth movements. Block 303 may encompass all aspects of target text selection and presentation. The target text may comprise one or more sentences, phrases, or other linguistic units of a complexity appropriate to the patient's current performance level.

In certain embodiments, the target text may be characterized by a set of complexity parameters comprising one or more of: a sentence length measured as a number of words or a number of syllables; a lexical complexity measured as a proportion of words below a defined word frequency threshold; a phonotactic complexity measured as a count or proportion of consonant clusters in onset or coda positions; a syntactic complexity measured as a number of clauses or a depth of syntactic embedding; and an entrainment rate measured as a speech rate in syllables per second at which the synthesized audio is generated. The complexity parameters of the target text presented in block 303 may correspond to the complexity determined in block 308 of a prior session or a prior trial within the current session.

In certain embodiments, the target text may be characterized by a set of complexity parameters comprising one or more of: a sentence length measured as a number of words or a number of syllables; a lexical complexity measured as a proportion of words below a defined word frequency threshold; a phonotactic complexity measured as a count or proportion of consonant clusters in onset or coda positions; a syntactic complexity measured as a number of clauses or a depth of syntactic embedding; and an entrainment rate measured as a speech rate in syllables per second at which the synthesized audio is generated. The complexity parameters of the target text presented in block 303 may correspond to the complexity determined in block 308 of a prior session or a prior trial within the current session.

Block 303 may further encompass receiving, via a clinician interface, one or more linguistic targets specifying characteristics of the target text, and generating the target text in accordance with those specified targets. A clinician (such as a speech-language pathologist) may, through a clinician-facing portal or application interface, specify one or more characteristics for the target text, including without limitation: targeted phoneme classes (e.g., fricatives, plosives, or vowels presenting difficulty for the patient); targeted lexical categories (e.g., open-class content words such as nouns and verbs, or closed-class function words); targeted syllabic complexity (e.g., monosyllabic versus polysyllabic words); targeted phonotactic complexity (e.g., onset clusters, coda clusters); or targeted semantic domains. These clinician-specified linguistic targets may guide the automatic generation or selection of target text sentences for the current or subsequent session, enabling the clinician to align therapy content with the patient's individualized treatment plan.

Block 303 may further encompass receiving a patient text preference and generating the target text based on that preference. In certain embodiments, the patient or the patient's caregiver may be allowed by the system to specify content preferences for the therapy scripts, such as personally meaningful topics (e.g., family members' names, favorite activities, familiar places). Personalized scripts may enhance patient engagement and may leverage the motivational salience of personally relevant content to facilitate practice and retention.

In certain embodiments, the one or more linguistic targets received via the clinician interface may specify one or more target phoneme classes, and the system may generate or select target text comprising words containing phonemes from the specified target phoneme classes at a frequency exceeding a defined minimum proportion of total phonemes in the target text. In certain embodiments, the system may automatically identify phoneme classes for which the patient demonstrates reduced synchronicity performance based on phoneme-level synchronicity metrics computed in block 306, and may increase the representation of those phoneme classes in subsequently generated target text without requiring explicit clinician specification.

In certain embodiments, the one or more linguistic targets received via the clinician interface may specify one or more target phoneme classes, and the system may generate or select target text comprising words containing phonemes from the specified target phoneme classes at a frequency exceeding a defined minimum proportion of total phonemes in the target text. In certain embodiments, the system may automatically identify phoneme classes for which the patient demonstrates reduced synchronicity performance based on phoneme-level synchronicity metrics computed in block 306, and may increase the representation of those phoneme classes in subsequently generated target text without requiring explicit clinician specification.

In certain embodiments, clinician input to the system may extend beyond specification of linguistic targets to include broader patient profile parameters that may shape the therapy configuration. For example, the patient's aphasia severity classification (e.g., mild, moderate, or severe), formal assessment scores (e.g., Aphasia Quotient from the Western Aphasia Battery-Revised), communication goals and functional priorities (e.g., family communication, workplace communication, community navigation), and relevant medical history (e.g., stroke chronicity, lesion location, or co-occurring motor speech disorder) may be entered or uploaded by a clinician through the clinician interface. These clinician-provided parameters may be used by the system to initialize appropriate default settings for initial target text complexity, entrainment mode selection, and rate parameters, providing a clinician-informed starting point for the adaptive algorithm that may be refined over subsequent sessions based on measured performance. All automatically generated target text scripts may further be reviewed, edited, and approved by the clinician before those scripts are presented to the patient, ensuring clinical appropriateness and alignment with the patient's individualized treatment plan. In certain embodiments, specific sentences may also be flagged by the clinician for exclusion if they are deemed clinically inappropriate or outside the patient's current functional communication needs.

In block 304, the synthesized speech 208 may be presented by the system to the patient: the synthesized audio may be played to the patient via the speaker 104 of the computing device 102, and the synthesized mouth movements may be displayed on the display 106 (in the audiovisual mode). The patient may be instructed to read the target text 109 aloud in synchronization with the synthesized audio and, where applicable, with the synthesized mouth movements.

Block 304 may encompass the full range of stimulus presentation parameters and adaptations. In certain embodiments, the synthesized audio may be played at a playback rate that increases over the course of therapy across sessions or within a session, such that the temporal demand on the patient's synchronization may progressively increase. For example, the system may begin with a comfortable, slow-to-moderate speech rate and may incrementally increase the rate as the patient's synchronicity measure improves over successive sessions. Rate manipulation may be used to probe the stability of the patient's predictive timing alignment, with the expectation that increased temporal demand may differentially impact synchronization performance and thereby identify the appropriate therapeutic challenge level for each patient.

Block 304 may further encompass stimulus fading, a procedure in which the amplitude of the synthesized audio (and, in the audiovisual mode, the amplitude or prominence of the synthesized visual stimulus) may be progressively attenuated across successive repetitions of the target text within a trial set, such that the patient's speech may transition from being externally cued (fully supported by the auditory and/or audiovisual entrainment model) to being independently generated without external support. The progressive attenuation may promote gradual internalization of speech-motor timing and may reduce reliance on the external self-cue over the course of the trial sequence. Stimulus fading may be implemented across any number of discrete amplitude levels or as a continuous monotonic attenuation function across repetitions, and the rate of attenuation may be adjusted based on the patient's performance. In an example three-level discrete fading sequence: the synthesized audio may be presented at 100% amplitude during the first repetition (full external support, patient speech fully cued); at an intermediate amplitude (for example, approximately 40% to 67% of the original amplitude, depending on the rate of fading selected) during a second repetition (partial support, patient begins internalizing timing); and at a minimal or zero amplitude (for example, approximately 0% to 33% of the original amplitude, or fully silent) during a third repetition (minimal or no support, patient may produce speech with largely or fully internalized timing). In an example continuous fading implementation, the amplitude of the synthesized stimulus may be linearly or exponentially attenuated from 100% at the onset of the first repetition to 0% at the conclusion of the final repetition, with the attenuation applied as a continuous gain function over the total playback duration, such that each successive moment of the stimulus may be slightly quieter than the preceding moment. In further discrete fading examples: a two-level sequence may present full support in the first repetition and no support in the second; a four-level sequence may present support levels at 100%, 67%, 33%, and 0% across four successive repetitions; or any other number of discrete amplitude steps may be used. In embodiments where stimulus fading is also applied to the visual channel, the synthesized mouth video may be progressively reduced in visual contrast, display brightness, or spatial prominence across repetitions in concert with the attenuation of the auditory channel. The specific fading schedule (the number of levels, the amplitude at each level, and the attenuation function) may be a configurable therapy parameter that may be set by the clinician or adjusted automatically by the system based on the patient's synchronicity performance. This fading approach may promote the transfer of speech production control from external self-cueing to self-initiated, independently generated speech, facilitating generalization of therapy gains beyond the entrainment context.

In certain embodiments, the method may comprise presenting the therapy session as a sequence of structured trials, each trial comprising a listening phase and an entrainment phase. In the listening phase, the synthesized audio may be played to the patient via the speaker while the patient attends to the stimulus without simultaneously speaking, and a record control on the computing device may be disabled during the listening phase. In the entrainment phase, the patient may activate the record control to initiate simultaneous playback of the synthesized audio and recording of the patient's speech output, and the target text may optionally be removed from the display during the entrainment phase such that the patient produces the target utterance from memory. In certain embodiments, the patient may listen to the synthesized audio a plurality of times during the listening phase before the entrainment phase begins.

In certain embodiments, the method may comprise presenting the therapy session as a sequence of structured trials, each trial comprising a listening phase and an entrainment phase. In the listening phase, the synthesized audio may be played to the patient via the speaker while the patient attends to the stimulus without simultaneously speaking, and a record control on the computing device may be disabled during the listening phase. In the entrainment phase, the patient may activate the record control to initiate simultaneous playback of the synthesized audio and recording of the patient's speech output, and the target text may optionally be removed from the display during the entrainment phase such that the patient produces the target utterance from memory. In certain embodiments, the patient may listen to the synthesized audio a plurality of times during the listening phase before the entrainment phase begins.

Block 304 may further encompass the structured trial interface through which the synthesized speech may be delivered to the patient during a therapy session. Each therapy trial may be organized into two sequential phases. In the first phase (a practice or listening phase) the target text may be displayed on the display 106 and the patient may press a Listen control (such as a button labeled “Listen” on the application interface) to initiate playback of the synthesized stimulus. The synthesized audio (and, in the audiovisual mode, the synthesized mouth video) may be played for the patient, and the patient may be directed to attend to the stimulus without simultaneously attempting to speak. In certain embodiments, the patient may listen to the stimulus a plurality of times during this phase (for example, two successive playback repetitions) before the active entrainment phase begins. During the listening phase, the record control (such as a “Record” button on the interface) may be disabled and unavailable for activation until listening playback has completed, preventing premature initiation of the entrainment recording phase before the patient has had an opportunity to attend to the model. In the second phase (the entrainment phase) the patient may activate the record control to initiate simultaneous playback of the synthesized stimulus and recording of the patient's own speech output. In certain embodiments, when the record control is activated, the textual display of the target sentence on the display 106 may be removed, requiring the patient to produce the target utterance from memory of the listened-to model rather than by reading from displayed text. Both the listen control and the record control may be available and active during the entrainment phase, enabling the patient to navigate the trial. In the audiovisual mode, the synthesized mouth video may be displayed on the display 106 after the record control is activated, presenting the patient with the synchronized audiovisual entrainment stimulus during the active recording phase. The patient may synchronize their speech and, in the audiovisual mode, their visible mouth movements with the synthesized audiovisual model during the entrainment phase, with the patient's audio output (and optionally video output) recorded for subsequent synchronicity analysis in block 306.

In block 305, the patient's speech output during the synchronization exercise may be recorded by the system—i.e., while the patient is reading the target text aloud in attempted synchronization with the synthesized audio. This recording may be captured by the microphone 105 of the computing device 102. In certain embodiments, the patient's mouth and facial movements during the entrainment phase may also be recorded by the system using the camera 103, providing both an audio recording and a video recording of the patient's speech production. The audio recording may capture the acoustic onset timing of the patient's speech production relative to the timing of the synthesized stimulus, providing the raw data needed for computation of the synchronicity measure in block 306. The video recording, where obtained, may capture the timing and appearance of the patient's lip and jaw movements during entrainment, enabling comparison of the patient's articulatory kinematics with those of the synthesized mouth model displayed during the audiovisual entrainment phase. In certain embodiments, visual articulatory timing features extracted from the patient's recorded video—such as lip aperture onset timing or jaw opening onset—may be incorporated into the synchronicity analysis of block 306 as supplementary measures of audiovisual entrainment quality. The recorded audio and video data from block 305 may be transmitted to the server 511 (via the communication network 510) for storage and optional clinician review through the provider portal.

In block 306, the synchronicity of the patient's speech output relative to the synthesized audio may be measured by the system. The synchronicity measure 209 may quantify the temporal alignment between the onset of the patient's speech and the onset of the synthesized model speech, corrected for device latency. The specific computation of the synchronicity measure is described in detail with respect to FIG. 4. In general, the synchronicity measure may comprise one or more of the following metrics: a signed timing offset (the difference between the patient's voiced onset time and the model's voiced onset time, corrected for device latency); a median timing offset computed across multiple repetitions of the target text (representing alignment accuracy); and a standard deviation of timing offsets (representing alignment stability or consistency). These metrics may collectively assess both the accuracy and the stability of the patient's synchronization with the entrainment stimulus.

In certain embodiments, the synchronicity measurement algorithm may compute a cross-correlation function between the time-domain or spectral representations of the entrainment stimulus and the patient attempt, and may identify the latency offset at which the cross-correlation is maximized. A synchronicity score may be derived from the magnitude of the cross-correlation peak at the optimal offset, with higher scores indicating greater acoustic similarity between the patient's attempt and the entrainment stimulus. In other embodiments, the synchronicity measurement may be performed in a feature space—for example, using mel-frequency cepstral coefficient (MFCC) sequences or learned speech embeddings—rather than in the raw time domain, providing robustness to minor timing variations and irrelevant acoustic differences.

The synchronicity measurement performed in block 306 may be implemented through signal processing algorithms operating on raw audio waveform data. The system may perform the following computational operations: (a) the embedded calibration tone (synchronizing tone 401) in the patient's recorded audio channel may be detected by applying a bandpass filter centered on the known tone frequency and identifying the temporal boundaries of the tone pulse, from which the device latency reference (d) may be computed as the temporal offset between the known playback time and the detected recording time of the calibration signal; (b) voiced onset detection may be performed on the synthesized model speech waveform and on the patient's recorded speech waveform using an acoustic onset detection algorithm that identifies the time point at which the amplitude envelope of the speech signal exceeds a threshold, yielding the model voiced onset time(s) and the patient voiced onset time (p) as scalar values; (c) the latency-adjusted patient onset time may be computed as (p−d) and the signed timing offset may be computed as (p−d)−s, producing a scalar metric with millisecond-level precision; and (d) signed offset values may be aggregated across multiple repetitions of the target text within a session to compute the median offset (alignment accuracy), the standard deviation of offsets (alignment stability), and, where word-level alignment is performed, the mean asynchrony per word and the overall phase alignment metric. These computed metrics may be stored in a data structure comprising an array of scalar values associated with session identifiers, trial identifiers, and timestamp metadata.

In certain embodiments, measuring the synchronicity may comprise: embedding a calibration tone of a known frequency and known duration in an audio channel at the beginning of each trial; detecting the calibration tone in the patient's recorded audio channel by applying a bandpass filter centered on the known frequency; computing a device latency value as a temporal offset between a known playback time of the calibration tone and a detected recording time of the calibration tone; detecting a voiced onset time of the synthesized audio; detecting a voiced onset time of the patient's recorded speech output; computing a latency-adjusted patient onset time by subtracting the device latency value from the patient's voiced onset time; and computing a signed timing offset as the difference between the latency-adjusted patient onset time and the voiced onset time of the synthesized audio. In certain embodiments, the signed timing offset may be computed for each of a plurality of repetitions of the target text within a session, and the synchronicity measure may comprise a median of the signed timing offsets across the plurality of repetitions and a standard deviation of the signed timing offsets across the plurality of repetitions.

In certain embodiments, measuring the synchronicity may comprise: embedding a calibration tone of a known frequency and known duration in an audio channel at the beginning of each trial; detecting the calibration tone in the patient's recorded audio channel by applying a bandpass filter centered on the known frequency; computing a device latency value as a temporal offset between a known playback time of the calibration tone and a detected recording time of the calibration tone; detecting a voiced onset time of the synthesized audio; detecting a voiced onset time of the patient's recorded speech output; computing a latency-adjusted patient onset time by subtracting the device latency value from the patient's voiced onset time; and computing a signed timing offset as the difference between the latency-adjusted patient onset time and the voiced onset time of the synthesized audio. In certain embodiments, the signed timing offset may be computed for each of a plurality of repetitions of the target text within a session, and the synchronicity measure may comprise a median of the signed timing offsets across the plurality of repetitions and a standard deviation of the signed timing offsets across the plurality of repetitions.

Block 306 may further encompass computation of word-by-word alignment (mean asynchrony) and overall phrase-level timing alignment (phase alignment), comparing the patient's production across the full sentence to the model's production. Mean asynchrony and phase alignment may be correlated but not identical: a patient may achieve good phase alignment (overall sentence duration matching the model) while exhibiting high mean asynchrony at the word level, revealing within-sentence timing irregularities that may be obscured by sentence-level analysis alone. Positive mean asynchrony may indicate that the patient's word onsets lag behind the corresponding model word onsets, consistent with reactive or delayed imitation; negative mean asynchrony may indicate anticipatory entrainment. Together, these complementary metrics may provide a more complete picture of the patient's synchronization profile than either metric alone.

The synchronicity metrics as a set may capture the full spectrum of entrainment behavior observed in patients, from true predictive entrainment through various degrees of reactive synchronization. These metrics may collectively inform both the real-time assessment of the patient's entrainment performance and the adaptive complexity determination of block 308. A patient demonstrating consistently low median offset, low standard deviation, and high word-by-word alignment across multiple repetitions of sentences at the current complexity level may trigger the system to advance to higher-complexity target text. Conversely, a patient demonstrating high median offset, high standard deviation, or poor word-by-word alignment may cause the system to maintain the current complexity or reduce it, ensuring that therapy may remain within the patient's zone of productive challenge.

In block 307, a report of the therapy session comprising the synchronicity measure 209 and the determined complexity for the subsequent target text may be generated by the system and transmitted to a clinician. The session report may be transmitted via the communication network (element 510 of FIG. 5) to a server (element 511) accessible by the clinician. The session report may be reviewed by the clinician through a web-based provider portal or a clinician application that may display the patient's session-by-session synchronicity trends, the complexity levels achieved, and the specific target texts presented. In certain embodiments, the report may further include the audio recording of the patient's speech output from block 305 and, in the audiovisual mode, the video recording of the patient's face. In further embodiments, the report may include automated speech analysis metrics such as words per minute, open-class word count, and fluency measures, providing the clinician with a comprehensive view of the patient's therapy progress.

In certain embodiments, the report may comprise one or more of: the synchronicity measure for each trial within the session; the determined complexity for the subsequent target text; an audio recording of the patient's speech output; a video recording of the patient's face during the entrainment exercise; the target text presented during the session; the difficulty level at the start and end of the session; automated speech analysis metrics comprising words per minute, open-class word count, closed-class word count, and open-to-closed class ratio; and session duration. The report may be transmitted via the communication network to a server accessible by the clinician through a web-based provider portal or a clinician application. In certain embodiments, the report may be generated automatically by the system upon completion of the therapy session without requiring patient or clinician initiation.

In certain embodiments, the report may comprise one or more of: the synchronicity measure for each trial within the session; the determined complexity for the subsequent target text; an audio recording of the patient's speech output; a video recording of the patient's face during the entrainment exercise; the target text presented during the session; the difficulty level at the start and end of the session; automated speech analysis metrics comprising words per minute, open-class word count, closed-class word count, and open-to-closed class ratio; and session duration. The report may be transmitted via the communication network to a server accessible by the clinician through a web-based provider portal or a clinician application. In certain embodiments, the report may be generated automatically by the system upon completion of the therapy session without requiring patient or clinician initiation.

The session report generation and transmission of block 307 may be performed automatically by the system's processor, which may aggregate the quantitative synchronicity metrics computed in block 306, the complexity parameters determined in block 308, the target text presented, and, in certain embodiments, the recorded audio and video data, into a structured digital report that may be transmitted via the communication network 510 to the server 511.

In block 308, a complexity for a subsequent target text may be determined by the system based on the synchronicity measure 209, and the subsequent target text may be generated at that complexity. The synchronicity measure 209 from the current session, or from a plurality of prior sessions, may be used to determine the appropriate level of linguistic complexity for the next therapy session's target text. In certain embodiments, the system may receive a synchronicity measure from a prior therapy session of the patient and may select the target text for a current or subsequent therapy session based on the synchronicity measure from the prior therapy session. In general, a synchronicity measure indicating strong alignment (i.e., low median timing offset and high alignment stability) may trigger an increase in complexity, while a synchronicity measure indicating poor alignment may trigger maintenance of the current complexity level or a decrease in complexity.

Block 308 may encompass all dimensions of complexity that may be adjusted by the system, including without limitation: sentence length (number of words or syllables per sentence); lexical complexity (proportion of low-frequency or morphologically complex words); phonotactic complexity (presence of onset and coda consonant clusters); syntactic complexity (clause structure, embedding depth); targeted linguistic features specified by a clinician (as described with respect to block 303); and entrainment rate (as described with respect to block 304).

In certain embodiments, the complexity determination may be further based on synchronicity measures accumulated across a plurality of prior therapy sessions, with recent sessions weighted more heavily than earlier sessions to reflect the patient's current performance trajectory. This longitudinal, recency-weighted adaptation may ensure that the system's difficulty progression is responsive to the patient's present functional state, rather than being dominated by early-therapy performance that may no longer reflect the patient's current ability. The longitudinal adaptation algorithm embodied in block 308 may operate as follows: at the conclusion of each therapy session, a session-level synchronicity summary may be computed by the system (for example, the median offset and standard deviation of offsets across all trials within the session, as well as mean asynchrony and phase alignment metrics). The current session's summary may then be incorporated by the system into a weighted performance estimate that combines current and historical session data, with more recent sessions assigned greater weight in the estimate than earlier sessions. This may be conceptually analogous to a Markov property in the sense that the system's adaptation decision may be primarily conditioned on the most recent state, while the full history of prior states may provide a secondary conditioning context.

In a first example implementation, a session-level complexity score may be computed by the system using an exponential moving average (EMA) of the synchronicity summary across sessions. Under the EMA approach, the estimated performance at session n may be computed as: EMA_n=alpha*S_n+(1−alpha)*EMA_{n−1}, where S_n is the synchronicity summary statistic for session n (for example, the median signed offset expressed as an absolute value, such that lower values may indicate better performance), EMA_{n−1} is the estimated performance from the prior session, and alpha is a smoothing factor (0<alpha<=1) that controls the degree of recency weighting. A higher alpha value may place greater weight on the most recent session; a lower alpha value may produce a smoother estimate that is less sensitive to single-session fluctuations. The alpha parameter may be configured by the clinician or system based on the patient's clinical profile and the desired responsiveness of the adaptation algorithm. In a second example implementation, a sliding window average may be computed by the system over the N most recent sessions (for example, N=3 or N=5), discarding sessions older than the window, such that the adaptation decision may be determined entirely by recent performance history without any influence from sessions outside the window. In a third example implementation, explicit decreasing weights may be assigned by the system to sessions in reverse chronological order—for example, assigning weight 1.0 to the most recent session, weight 0.5 to the session before that, weight 0.25 to the session before that, and so on—computing a weighted average synchronicity summary from which the complexity determination may be derived. In a fourth example implementation, a threshold-crossing rule may be used by the system: if the patient's synchronicity summary has met or exceeded a specified performance threshold (for example, median offset within ±200 ms and standard deviation below 150 ms) for each of the M most recent consecutive sessions (for example, M=2 or M=3), the complexity level may be advanced by the system; if the threshold has not been met for K consecutive sessions (for example, K=2), the complexity level may be reduced by the system; and otherwise the complexity level may be held constant. These four example implementations are non-limiting; any suitable recency-weighted or session-history-based algorithm for deriving a complexity determination from accumulated synchronicity data may fall within the scope of block 308.

In certain embodiments, the complexity determination may comprise comparing the synchronicity measure against one or more predefined threshold values stored in a patient profile data structure. The predefined threshold values may comprise an advancement threshold and a regression threshold. If the synchronicity measure meets or exceeds the advancement threshold for a configurable number of consecutive attempts or consecutive sessions, the complexity for the subsequent target text may be increased. If the synchronicity measure falls below the regression threshold for a configurable number of consecutive attempts or consecutive sessions, the complexity for the subsequent target text may be decreased. In certain embodiments, the advancement threshold may comprise a median offset within a defined range of zero milliseconds and a standard deviation of offsets below a defined maximum value. In certain embodiments, the complexity for the subsequent target text may be represented as a complexity parameter vector comprising values for one or more of: sentence length measured in words or syllables, lexical frequency band, phonotactic complexity index, syntactic embedding depth, and entrainment rate.

In certain embodiments, the complexity determination may comprise comparing the synchronicity measure against one or more predefined threshold values stored in a patient profile data structure. The predefined threshold values may comprise an advancement threshold and a regression threshold. If the synchronicity measure meets or exceeds the advancement threshold for a configurable number of consecutive attempts or consecutive sessions, the complexity for the subsequent target text may be increased. If the synchronicity measure falls below the regression threshold for a configurable number of consecutive attempts or consecutive sessions, the complexity for the subsequent target text may be decreased. In certain embodiments, the advancement threshold may comprise a median offset within a defined range of zero milliseconds and a standard deviation of offsets below a defined maximum value. In certain embodiments, the complexity for the subsequent target text may be represented as a complexity parameter vector comprising values for one or more of: sentence length measured in words or syllables, lexical frequency band, phonotactic complexity index, syntactic embedding depth, and entrainment rate.

The longitudinal adaptation engine may be designed to handle multiple clinically relevant scenarios. In the scenario where a patient demonstrates a plateau (sustained performance at the same complexity level across multiple sessions without continued improvement in synchronicity), a complexity perturbation may be applied by the system, such as introducing a brief period of increased or decreased difficulty, to probe whether the plateau reflects consolidation of the current skill level or a genuine performance ceiling. In the scenario where a patient resumes therapy after an interruption of one or more missed sessions, a conservative recency-weighting policy may be applied by the system that re-initializes the EMA or sliding window based only on the sessions from the current therapy episode, rather than sessions from prior episodes, to avoid overfitting complexity selection to stale historical data. In the scenario where a patient's performance degrades across sessions (for example, due to clinical regression, fatigue, or health events), the sustained recency-weighted average may reflect the degradation and may trigger a downward complexity adjustment, ensuring that therapy difficulty may track the patient's current functional level in both directions.

Block 308 may further encompass automatic generation of the subsequent target text at the determined complexity. In certain embodiments, a language model or a text generation algorithm may be employed by the system to automatically produce novel sentences satisfying the specified complexity parameters, optionally filtered to align with any clinician-specified linguistic targets or patient-specified content preferences. The generated target text may be stored and loaded for the subsequent therapy session (block 303 of the next session cycle), and may be previewed and approved by the clinician before being presented to the patient.

In certain embodiments, generating the subsequent target text may comprise providing the determined complexity parameters and any clinician-specified linguistic targets as constraints to a language model, and receiving from the language model one or more candidate sentences satisfying the constraints. The language model may comprise a large language model accessed via a network API call to a remote server, or a locally stored text generation model. The candidate sentences may be filtered by the system to exclude sentences containing vocabulary outside a defined frequency band, sentences exceeding a maximum syllable count, or sentences containing phonotactic structures not targeted by the current therapy phase. In certain embodiments, the generated subsequent target text may be stored in a target text queue associated with the patient's profile and may be presented to the patient in a subsequent therapy session without requiring real-time generation during the session.

In certain embodiments, generating the subsequent target text may comprise providing the determined complexity parameters and any clinician-specified linguistic targets as constraints to a language model, and receiving from the language model one or more candidate sentences satisfying the constraints. The language model may comprise a large language model accessed via a network API call to a remote server, or a locally stored text generation model. The candidate sentences may be filtered by the system to exclude sentences containing vocabulary outside a defined frequency band, sentences exceeding a maximum syllable count, or sentences containing phonotactic structures not targeted by the current therapy phase. In certain embodiments, the generated subsequent target text may be stored in a target text queue associated with the patient's profile and may be presented to the patient in a subsequent therapy session without requiring real-time generation during the session.

Based on the synchronicity score computed for each patient attempt, an adaptive content selection algorithm may be applied by the system to determine the practice text and difficulty level for the next stimulus presentation. In certain embodiments, a predefined advancement criterion may be applied by the adaptive algorithm: if the patient's synchronicity score meets or exceeds a defined threshold (e.g., a threshold specified in the patient profile data structure) for a configurable number of consecutive attempts, the patient may be advanced by the system to a higher difficulty level within the current session. Conversely, if the synchronicity score falls below a defined regression threshold for a configurable number of consecutive attempts, the difficulty level may be reduced by the system or previously mastered content may be returned to. In certain embodiments, difficulty level may be operationalized as a function of one or more of the following: phoneme complexity; utterance length; speech rate of the entrainment stimulus; prosodic variability; or semantic complexity of the practice text. The specific relationship between difficulty level and these parameters may be defined in a difficulty progression data structure associated with the patient's therapy protocol.

Upon completion of a therapy session—whether by exhaustion of the scheduled session duration, completion of a target number of practice attempts, or patient-initiated session termination—the session log may be compiled by the system as described herein and stored to a session history database associated with the patient's account. In certain embodiments, a session completion confirmation may be displayed to the patient, which may include a summary of session performance (e.g., mean synchronicity score for the session, number of attempts completed, and difficulty level achieved), presented in a patient-accessible format designed to provide motivational feedback. In certain embodiments, the session results may additionally be evaluated by the system against the criteria for scheduling adjustment and difficulty parameter update, and the patient profile data structure may be updated accordingly before the patient's next session.

FIG. 4 illustrates an example of audio waveforms and synchronicity measurements that may be used to assess the patient's speech synchronization performance during a mirror speech entrainment therapy session. FIG. 4 may depict a timeline view of three acoustic channels: the synchronization tone channel (element 401), the model speech channel (element 402), and the patient speech channel (element 403). The offset measurement between these channels (elements 404 and 409) may provide the quantitative synchronicity measure 209.

The synchronizing tone 401 may be an embedded calibration signal—in the illustrated example, a 1,000 millisecond (1-second) pure-tone beep—that may be recorded into the patient's recording channel at the beginning of each trial. The synchronizing tone 401 may serve as a timing reference that enables computation of the device latency reference (d). Because the patient's recording may be captured by the microphone 105 of the computing device 102, a measurable latency (d) may exist between the time at which the synthesized audio is played through the speaker 104 and the time at which that audio is detected by the microphone 105 and recorded in the patient's channel. The pulse of synchronization tone 405 may be the acoustic pulse corresponding to the synchronizing tone 401 as it appears in the patient's recorded channel. The center point of tone 406 may mark the temporal center of the synchronizing tone pulse, which may provide the reference point for computing the device latency correction value (d).

The modeled voice output 402 may represent the waveform of the synthesized model speech (the synthetic speech audio 204) as it appears in the recording channel, with its onset occurring at the synthesized speech onset time 410, denoted as(s). The synthesized speech waveform 407 may depict the full temporal extent of the model's synthesized speech signal for the trial. The model voiced onset time(s) may be the time at which the synthesized model voice begins phonation for the target sentence.

The patient voice output 403 may represent the waveform of the patient's live speech production during the synchronization exercise, as captured by the microphone 105. The patient speech waveform 408 may depict the temporal extent of the patient's speech signal. The onset time for patient voice 414, denoted as (p), may be the time at which the patient begins phonation.

The offset measurement 404 may be the primary synchronicity metric, computed according to the formula: Signed offset=(p−d)−s, where p may be the patient's voiced onset time, d may be the device latency reference, and s may be the model voiced onset time. The term (p−d) may represent the latency-adjusted participant onset time 413: the patient's voiced onset time corrected for device latency. The difference between the latency-adjusted participant onset (p−d) and the model voiced onset(s) may yield the signed timing offset 409.

The device latency reference (d) may correct for the round-trip acoustic delay between the playback of the synthesized audio through the speaker 104 and the detection of that audio signal by the microphone 105, which may be inherent to the patient's recording configuration. In the illustrated example, the latency reference (d) may be computed from a calibration signal (a synchronizing tone embedded at the beginning of each trial) and may correspond to the temporal center of the synchronizing tone pulse as it appears in the patient's recording channel. The use of the temporal center of the tone pulse as the reference point may be selected for practical convenience: the center of a known-duration tone pulse may be readily identifiable in a waveform display and may be robust to slight variations in tone onset detection caused by microphone noise or recording artifacts. Alternative latency reference points may be used in other embodiments. For example, in certain embodiments, the latency reference may be computed from the onset of the synchronizing tone pulse (the leading edge at which the tone transitions from silence to sustained phonation) rather than from the center of the tone, providing a reference point that may more closely reflect the acoustic propagation delay of the onset of any speech signal. In further embodiments, the latency correction may additionally account for human motor response time (the irreducible neural and motor delay between the patient's perception of the model onset and the initiation of the patient's articulatory movement) such that the corrected offset may more precisely isolate the component of the timing difference attributable to synchronization accuracy rather than to inherent physiological latency. In certain embodiments, the latency correction value may be established using an empirical device calibration procedure performed before each session, in which the system may play a series of calibration signals and may measure the round-trip acoustic delay directly from the recorded waveform. In other embodiments, a population- or device-level average latency value may be used as an approximation of (d), avoiding the need for a per-session calibration tone altogether and instead relying on a stored device latency constant. In further embodiments, the synchronizing calibration signal need not be a pure-tone beep; alternative calibration signals may include a broadband noise burst, a click, a speech-like onset transient, or any other signal with a clearly detectable temporal boundary in the recorded waveform. The selection of the specific latency correction approach and calibration signal may be a matter of implementation choice, and all such approaches may fall within the scope of the disclosure.

The measured offset 409 may represent the signed temporal offset between the patient's latency-adjusted speech onset and the model's speech onset. Positive offset values may indicate delayed shadowing or reactive imitation, in which the patient begins phonation after the model's onset (a pattern consistent with a patient who may be reacting to and following the model rather than entraining predictively to it). Negative offset values may indicate anticipatory alignment, in which the patient begins phonation slightly before the model's onset (a pattern characteristic of true predictive entrainment in which the patient may have internalized the model's timing sufficiently to begin speaking slightly ahead of the modeled stimulus). A zero or near-zero offset may indicate optimal synchronization, in which the patient's speech onset may be temporally aligned with the model's onset. Progressively negative offsets across sessions may reflect the patient's progressive internalization of the entrainment model's timing, representing the desired therapeutic trajectory from reactive shadowing toward predictive co-production.

In the illustrated example, the device latency reference (d) may occur at approximately 500 ms on the timeline. The model voiced onset(s) may occur at approximately 2,000 ms. The patient's voiced onset (p) may occur at approximately 4,200 ms. The latency-adjusted patient onset (p−d) may therefore be approximately 3,700 ms. The resulting signed offset may be approximately 3,700−2,000=+1,700 ms, indicating delayed shadowing—reactive imitation with a positive offset—consistent with a patient who may still be in early stages of synchronization training. It will be appreciated that this example may represent one end of the observed behavioral spectrum. In patients with more advanced synchronization, the signed offset may be in the range of a few hundred milliseconds positive (moderate delayed shadowing consistent with reactive synchronization to a concurrent model), near zero milliseconds (optimal temporal alignment with the model, characteristic of well-entrained production), or slightly negative (anticipatory alignment, indicating internalization of the model's timing and production of speech slightly ahead of the model onset). In clinical use, offsets across this full range—from large positive values characteristic of patients in early therapy stages through near-zero and negative values characteristic of well-entrained patients—may be expected and may all be captured and interpreted by the synchronicity measure. The trajectory of offset values across sessions may be particularly informative: a patient whose median offset decreases (becomes less positive or more negative) across sessions may demonstrate progressive improvement in temporal entrainment. Plateau or reversal of this trajectory may trigger adaptive complexity adjustments as described with respect to block 308.

The synchronicity measure 209 derived from these measurements may comprise one or more of the following:
Alignment accuracy (median offset): The median value of the signed offset computed across multiple repetitions of the target sentence within a session. A median offset approaching zero milliseconds may indicate high alignment accuracy. This metric may be the primary summary measure of a patient's synchronization accuracy for a given session.
Alignment stability (standard deviation of offsets): The standard deviation of the signed offset computed across repetitions. A low standard deviation may indicate consistent, stable synchronization; a high standard deviation may indicate variable or erratic synchronization, which may reflect difficulty with temporal prediction or fluctuating engagement with the entrainment model. Alignment stability may provide information about the reliability and robustness of the patient's entrainment that is complementary to, and not captured by, the alignment accuracy metric alone.
Mean asynchrony: The mean absolute or signed offset computed on a word-by-word basis, comparing each word's onset time in the patient's production to the corresponding word's onset time in the model's production, providing a fine-grained measure of within-sentence synchronization. Mean asynchrony may be particularly sensitive to within-sentence timing drift, in which a patient may achieve good sentence-level onset alignment but may fall progressively out of temporal alignment with the model across the interior of the sentence.
Phase alignment: The overall sentence-level alignment metric comparing the onset time, offset time, and total duration of the patient's sentence production to those of the model's production. A patient who produces the sentence at the same total duration and with matched onset and offset times as the model may be understood to achieve high phase alignment, regardless of finer within-sentence timing variation.

These synchronicity metrics may collectively inform both the real-time assessment of the patient's entrainment performance and the adaptive complexity determination of block 308.

The complexity determination described herein may use quantitative synchronicity metrics—including median offset in milliseconds, standard deviation of offsets, mean asynchrony per word, and phase alignment—computed through the signal processing algorithms of block 306, and may apply a decision function with numerical thresholds to those metrics to produce a complexity parameter set for subsequent target text generation.

FIG. 5 illustrates a computing environment 500 suitable for implementing the systems and methods disclosed herein. The computing environment 500 may comprise a user device 501, one or more servers (including server 511), and a communication network 510 connecting the user device 501 to the servers. The elements of FIG. 5 may be implemented using any suitable computing architecture, including cloud computing instances, bare-metal servers, virtual machines, or any combination thereof, as further described below. FIG. 5 is illustrative and non-limiting; the particular deployment topology may be a matter of implementation choice and may not affect the logical operation of the disclosed systems and methods.

The user device 501 may be any computing device suitable for use by a patient during a therapy session, including without limitation a smartphone, tablet computer, laptop computer, desktop computer, or a dedicated therapeutic device. In certain embodiments, the user device 501 may be a mobile device operated by the patient in a home setting, enabling flexible access to MSE therapy outside of clinical appointments. The user device 501 may comprise a processor 502, a non-transitory computer-readable medium 503, a communication system 504, a speaker 505, a microphone 506, a camera 507, and a display 508. The processor 502 may be a general-purpose processor, application-specific integrated circuit (ASIC), graphics processing unit (GPU), or other processing hardware suitable for executing stored instructions. The processor 502 may include multiple processing cores and may employ hardware accelerators for computationally intensive operations such as neural network inference.

The non-transitory computer-readable medium 503 may store instructions 509 for mirror speech entrainment therapy. The instructions 509 may comprise any or all of the method steps of FIG. 3 implemented as executable code: instructions to obtain a voice sample of the patient; instructions to synthesize audio of target text in the patient's own voice using the auditory speech synthesis model 203; instructions to present the target text to the patient via the display 508; instructions to instruct the patient to read the target text aloud in synchronization with the synthesized audio; instructions to play the synthesized audio via the speaker 505; instructions to record the patient's speech output via the microphone 506; instructions to measure the synchronicity of the patient's speech output relative to the synthesized audio; instructions to determine a complexity for a subsequent target text based on the synchronicity measure; and instructions to generate the subsequent target text at the determined complexity. In certain embodiments, the non-transitory computer-readable medium 503 may further store instructions to obtain a video sample of the patient via the camera 507, to synthesize mouth movements of the patient producing the target text using the visual speech synthesis model 206, and to display those synthesized mouth movements on the display 508 during the entrainment exercise.

The user device 501, when configured with the instructions 509 for mirror speech entrainment therapy, may integrate the processor 502, the speaker 505, the microphone 506, the camera 507, and the display 508 to enable the operations described herein. Specifically, the microphone 506 may serve a dual function: it may capture the patient's voice sample for speaker embedding extraction and voice cloning (block 301), and it may simultaneously record the patient's live speech output during the entrainment exercise for synchronicity measurement (block 305), including capturing the embedded calibration tone used for device latency correction. The speaker 505 may deliver the synthesized self-voice audio stimulus while the microphone 506 may concurrently record the patient's speech and the calibration tone, creating the multi-channel recording environment that may be required for latency-calibrated synchronicity computation. The camera 507 may capture the patient's facial appearance for visual speech synthesis model conditioning. The display 508 may simultaneously present the target text and the synthesized mouth movements, providing the dual visual stimulus that may be required for audiovisual entrainment.

In certain embodiments, the user device 501 may constitute a computing device for providing mirror speech entrainment-based speech therapy, the computing device comprising: the processor 502; the display 508; the speaker 505; and the non-transitory computer-readable medium 503 storing instructions 509 executable by the processor 502 to obtain a voice sample of a patient comprising audio of the patient speaking, synthesize audio of a target text read in a voice matching the patient's own voice using the voice sample, present the target text to the patient via the display 508, instruct the patient to read the target text aloud in synchronization with the synthesized audio, play the synthesized audio to the patient via the speaker 505, record the patient's speech output during the synchronization, measure a synchronicity of the patient's speech output relative to the synthesized audio, determine a complexity for a subsequent target text based on the synchronicity measure, and generate the subsequent target text based on the determined complexity. In certain embodiments, the computing device may further comprise the camera 507, and the instructions 509 may be further executable to obtain a video sample of the patient via the camera 507, synthesize mouth movements of the patient producing the target text synchronized with the synthesized audio using the video sample, and display the synthesized mouth movements to the patient via the display 508 during the synchronization. In certain embodiments, the non-transitory computer-readable medium 503 may constitute a non-transitory computer-readable medium storing instructions executable to perform each of the foregoing operations.

In certain embodiments, the user device 501 may constitute a computing device for providing mirror speech entrainment-based speech therapy, the computing device comprising: the processor 502; the display 508; the speaker 505; and the non-transitory computer-readable medium 503 storing instructions 509 executable by the processor 502 to obtain a voice sample of a patient comprising audio of the patient speaking, synthesize audio of a target text read in a voice matching the patient's own voice using the voice sample, present the target text to the patient via the display 508, instruct the patient to read the target text aloud in synchronization with the synthesized audio, play the synthesized audio to the patient via the speaker 505, record the patient's speech output during the synchronization, measure a synchronicity of the patient's speech output relative to the synthesized audio, determine a complexity for a subsequent target text based on the synchronicity measure, and generate the subsequent target text based on the determined complexity. In certain embodiments, the computing device may further comprise the camera 507, and the instructions 509 may be further executable to obtain a video sample of the patient via the camera 507, synthesize mouth movements of the patient producing the target text synchronized with the synthesized audio using the video sample, and display the synthesized mouth movements to the patient via the display 508 during the synchronization. In certain embodiments, the non-transitory computer-readable medium 503 may constitute a non-transitory computer-readable medium storing instructions executable to perform each of the foregoing operations.

The non-transitory computer-readable medium 503 may store, in addition to executable instructions, the following data structures integral to the operation of the disclosed system: (a) trained neural network model parameters comprising speaker encoder weight matrices, TTS decoder weight matrices, vocoder weight matrices, and (where visual synthesis is employed) visual speech synthesis model weight matrices, each being a multi-dimensional numerical array representing learned transformations that may be optimized through training on speech data; (b) patient-specific speaker embedding vectors, each being a fixed-dimensional numerical vector representing a particular patient's speaker identity in a learned embedding space, which may be stored in association with a patient identifier and may be used to condition the synthesis model for that patient; (c) complexity parameter vectors, each being a structured numerical record comprising values for sentence length, lexical frequency band, phonotactic complexity index, syntactic embedding depth, and entrainment rate, which may be stored in association with a patient identifier and a session identifier; and (d) synchronicity metric histories, each being a time-indexed array of scalar synchronicity values (median offset, standard deviation, mean asynchrony, phase alignment) that may be accumulated across therapy sessions for a given patient, stored in association with session identifiers and timestamp metadata.

The communication system 504 may provide network connectivity for the user device 501, supporting one or more of Wi-Fi, Bluetooth, cellular (3G, 4G, 5G), or other network communication protocols. The communication system 504 may enable the user device 501 to transmit patient data (including recorded speech, computed synchronicity measures, and session reports) to and from the server 511 via the communication network 510.

The stored model 515 may represent one or more machine learning models stored in accessible memory—either on the user device 501 or on the server 511 (or on multiple servers)—and may be used to perform the synthesis operations described with respect to block 302 of FIG. 3 and elements 203 and 206 of FIG. 2. In a first example configuration, both the auditory speech synthesis model 203 and the visual speech synthesis model 206 may be stored as elements of the stored model 515 on the server 511. In this configuration, the patient's audio and video samples may be transmitted by the user device 501 to the server 511 via the communication network 510, model inference may be performed by the server 511 to generate the synthetic speech audio 204 and the synthetic speech video 207, and the synthesized outputs may be transmitted back to the user device 501 for presentation to the patient. In a second example configuration, one model (e.g., the auditory speech synthesis model 203) may be stored on the server 511 while the other (e.g., the visual speech synthesis model 206) may be stored on a separate server. In a third example configuration, one or both models may be stored locally on the user device 501 as part of the stored model 515, enabling model inference to be performed on-device without requiring network connectivity during the synthesis step. This third configuration may be advantageous for patients in low-connectivity environments or where minimizing latency may be desired. Each of these configurations may fall within the scope of the disclosure.

The server 511 may comprise a processor 512, a non-transitory computer-readable medium 514, a communication system 513, and the stored model 515. The processor 512 may comprise multiple processing cores and hardware accelerators suitable for running neural network inference at the scale that may be required by the auditory speech synthesis model 203 and the visual speech synthesis model 206. The non-transitory computer-readable medium 514 may store model parameters for the stored model 515, patient-specific model instances or voice embeddings associated with individual patients, session report data, historical synchronicity measures, and any other data needed to support the server-side operations of the MSE system. The stored model 515 on the server 511 may comprise the auditory speech synthesis model 203 alone, the visual speech synthesis model 206 alone, or both, depending on the deployment configuration selected. The communication system 513 may connect the server 511 to the communication network 510 and thereby to the user device 501.

In the computing environment 500, the communication network 510 may connect the user device 501 and the server 511. The communication network 510 may comprise the Internet, a cellular network, a local area network (LAN), a wide area network (WAN), a virtual private network (VPN), or any combination thereof. The communication network 510 may facilitate transmission of patient audio and video samples from the user device 501 to the server 511 for synthesis, transmission of synthesized audio and video outputs from the server 511 to the user device 501 for presentation, and transmission of session report data and synchronicity measures from the user device 501 to a clinician-accessible server for review.

The mobile device computing environment, including the user device 501, may include additional subcomponents. The memory of the user device may be volatile (RAM) or non-volatile (ROM, flash storage, SSD) and may store user profiles, therapy settings, patient-specific voice embeddings, and application data. The display 508 may be a touch screen that may also function as a user input device. The communication system 504 may support end-to-end encryption to ensure data privacy and compliance with applicable healthcare data protection requirements. In certain embodiments, therapy progress data may be synchronized by the user device 501 across multiple devices, enabling continuity of therapy sessions even when a patient switches between a smartphone, tablet, and other devices.

The server 511 may host a web-based provider portal accessible by clinicians (including speech-language pathologists) to remotely monitor patient progress, review session reports generated in block 307 of FIG. 3, configure linguistic target parameters, adjust complexity thresholds, and approve subsequent target text generated in block 308. In certain embodiments, the server 511 may be integrated with an electronic medical records (EMR) system to provide seamless access to patient therapy data. The server 511 may also be connected to a cloud-based large language model or text generation service for generating target text sentences as described with respect to block 308. In further embodiments, patient-specific model instances may be stored by the server 511—for example, a voice embedding or fine-tuned voice synthesis model trained on a particular patient's voice samples—enabling highly personalized synthesis that may improve in fidelity as more voice sample data is accumulated over the course of therapy.

All references to a “processor” herein may encompass general-purpose processors, multi-core processors, graphics processing units (GPUs), tensor processing units (TPUs), field-programmable gate arrays (FPGAs), application-specific integrated circuits (ASICs) optimized for machine learning inference, and any combination of the foregoing. All references to a “non-transitory computer-readable medium” may encompass solid-state drives (SSDs), flash memory, read-only memory (ROM), magnetic storage, optical storage, and any other non-transitory medium suitable for storing executable instructions. The particular computing hardware employed may be a matter of implementation choice and may not affect the logical operation of the disclosed systems and methods.

The following describes example results from feasibility studies conducted to assess the feasibility, efficacy, and user experience of mirror speech entrainment therapy using systems and methods as described herein. These studies were designed as single-arm, within-subjects controlled feasibility investigations, enrolling a small number of participants with confirmed diagnoses of nonfluent aphasia who were more than six months post-stroke. Each participant may have completed multiple therapy sessions using the MSE system, with sessions conducted via a mobile device platform. Performance was assessed using quantitative lexical and fluency metrics derived from connected speech samples collected at baseline and post-treatment time points. The experimental protocols and outcome measures described herein do not limit the scope of the disclosure.

The theoretical framework for MSE, described above in the overview of this Detailed Description, may integrate the Self-Attention Network (SAN), the Self-Reference Effect, Speech Entrainment, Script-based Intervention, and Principles of Experience-Dependent Neuroplasticity. The feasibility studies described below were designed to test the core prediction of this framework: that a self-referential entrainment stimulus—derived from the patient's own voice—may produce greater gains in lexical retrieval and speech motor fluency than a non-personalized entrainment stimulus.

Three feasibility studies, each employing a single-arm, within-subjects controlled design, were designed to test MSE across conditions and participant populations. Study 1 enrolled four English-speaking persons with aphasia (PWA) who were more than six months post-stroke, each sequentially receiving MSE-audio (auditory-only MSE) and MSE-video (audiovisual MSE) treatment conditions. Study 2 enrolled four additional PWA and compared MSE-video (the audiovisual MSE condition using a synthesized version of the patient's own voice and face) against traditional audiovisual speech entrainment (using a non-personalized external audiovisual model) within the same participants using a counterbalanced design. Study 3 compared MSE-audio (auditory-only MSE using the patient's own cloned voice) against traditional audio speech entrainment (using a non-personalized external voice model). Treatments within each study were counterbalanced across participants to control for order effects. Speech rate and accuracy were assessed at three time points: baseline (T1, prior to treatment), post-treatment one (T2, following the first treatment condition), and post-treatment two (T3, following the second treatment condition). Participant eligibility required a confirmed diagnosis of nonfluent aphasia, stroke chronicity of greater than six months at enrollment, English as the primary language, and no active expressive language treatment outside of the study. A one-week washout period was implemented between conditions to reduce carryover effects.

A detailed report of outcomes is available for participant MS2. MS2 is a 38-year-old, right-handed female and native speaker of English who experienced a left-hemisphere ischemic stroke on May 17, 2024. During acute hospitalization, speech-language pathology services focused on communication recovery were received. At the time of the study, she was engaged in speech therapy addressing dysphagia management; no additional speech-language treatment addressing expressive language was provided concurrently with study participation. MS2's highest level of education is a bachelor's degree. No history of developmental language or literacy difficulties and no prior neurological, psychiatric, or cognitive disorders were reported. Informed consent was obtained and the study was approved by the Institutional Review Board at the University of South Florida (IRB Protocol #007481).

Baseline language function was characterized using the Western Aphasia Battery-Revised (WAB-R; Kertesz, 2006), which includes measures of spontaneous speech, repetition, and naming. Spontaneous speech was elicited using the WAB-R Picnic Scene description task. Additional connected speech samples were elicited using standardized picture description stimuli, including the Cookie Theft (Goodglass et al., 2001), Cat Rescue, and Broken Window scenes (Nicholas & Brookshire, 1993). Speech samples were digitally recorded and transcribed using standardized CHAT-format transcription procedures. Transcripts were processed using CLAN (MacWhinney, 2000) and BatchAlign automated alignment software (Lanski et al., 2022) to extract quantitative lexical and fluency metrics.

Primary lexical outcome measures included: (1) open-class word count, defined as the total count of content words (nouns, verbs, adjectives, and adverbs) produced in connected speech samples; (2) closed-class word count, defined as the total count of function words (articles, prepositions, and pronouns) produced in connected speech samples; and (3) open-to-closed class ratio, defined as the ratio of open-class word count to closed-class word count, providing a measure of the relative proportion of semantically meaningful lexical content in spontaneous connected speech. Primary fluency outcome measures included: words per minute (WPM), calculated as total words divided by speech duration, providing a measure of temporal speech production efficiency and motor execution fluency. These measures may collectively quantify lexical retrieval capacity, speech production efficiency, and speech motor fluency.

Speech entrainment was performed using two entrainment conditions delivered via a mobile device platform. In the Mirror Speech Entrainment (MSE) condition, speech stimuli were presented using a synthesized voice model derived from the participant's own voice characteristics, as described with respect to the auditory speech synthesis model 203 and the pipeline of FIG. 2. In the Traditional Speech Entrainment (TSE) condition, identical textual stimuli were presented using a non-personalized external voice model that did not incorporate the participant's own voice characteristics. In both conditions, the participant listened to and repeated sentences while attempting to align timing, articulation, and prosody with the presented model. Pre-condition and post-condition speech samples were collected using identical picture description procedures at each assessment time point.

Quantitative analysis of connected speech revealed differential effects across entrainment conditions. Mirror Speech Entrainment was associated with increased open-class word production. Mean open-class word count increased from 29.5 words at pre-condition assessment to 81.0 words at post-condition assessment, representing an increase of 51.5 open-class words following MSE. In contrast, Traditional Speech Entrainment was associated with reduced open-class word production: mean open-class word count decreased from 68.5 words at pre-condition assessment to 38.5 words at post-condition assessment, representing a reduction of 30.0 words. Open-class words may represent semantically meaningful lexical items and may reflect access to lexical content during spontaneous speech production; the increase observed following MSE and not following TSE may suggest a differential effect of the self-referential entrainment model on lexical retrieval in connected speech.

Lexical efficiency, measured as the open-to-closed class ratio, remained stable and showed a slight increase following Mirror Speech Entrainment, with the open-to-closed class ratio increasing from 1.28 pre-condition to 1.30 post-condition. In contrast, Traditional Speech Entrainment was associated with a reduction in lexical efficiency, with the open-to-closed class ratio decreasing from 1.35 pre-condition to 1.16 post-condition. Maintenance of a high open-to-closed class ratio following MSE may indicate that the patient's proportional production of semantically meaningful content words was preserved or modestly enhanced, consistent with improved lexical access rather than a nonspecific increase in all word types.

Speech fluency, measured using words per minute (WPM), increased following Mirror Speech Entrainment. Mean speech rate increased from 199.9 WPM pre-condition to 270.4 WPM post-condition, representing an increase of 70.5 WPM following MSE. In contrast, Traditional Speech Entrainment was associated with reduced speech fluency: mean speech rate decreased from 280.4 WPM pre-condition to 232.3 WPM post-condition, representing a reduction of 48.1 WPM. Speech rate may provide an objective measure of temporal speech production performance and motor execution efficiency. The increase in speech rate following MSE, contrasted with the decrease following TSE in the same participant within a closely proximate timeframe, may be consistent with a differential effect of the personalized, self-referential MSE stimulus on speech motor fluency.

These example results may suggest that Mirror Speech Entrainment may be associated with increased open-class word production and increased speech rate in spontaneous, independent connected speech tasks performed without an external model present, providing evidence of generalization of treatment effects beyond the entrainment context itself. These results may be consistent with the feasibility of MSE as a tool for improving speech production in persons with nonfluent aphasia, and may be consistent with the theoretical prediction that a self-referential entrainment stimulus—derived from the patient's own voice and presenting a corrected, fluent version of the patient's speech—may contribute to greater gains in lexical retrieval and speech motor fluency than a traditional, non-personalized entrainment approach.

User feedback collected from an initial participant (n=1) using a structured affect rating scale indicated positive user experience with the MSE system. Rated dimensions included comfort, enjoyment, perceived naturalness, voice similarity, pride, excitement, discomfort, unease, and worry, assessed on a scale from 1 (low) to 5 (high). The participant rated naturalness at 5.0, voice similarity at 5.0, and enjoyment at 5.0, indicating a highly positive subjective experience of the personalized voice clone. Ratings for discomfort, unease, and worry were each 1.0, indicating an absence of negative affect associated with the self-referential synthesis experience. Ratings for pride and excitement were each 3.0. These user experience data may suggest that patients may experience MSE as comfortable, enjoyable, and natural, and may not indicate aversive reactions to hearing and seeing a synthesized version of their own voice and appearance—a result that may be consistent with the predicted salience and engagement benefits of the self-referential stimulus design.

In the foregoing specification, implementations of the disclosure have been described with reference to specific example implementations thereof. It will be evident that various modifications may be made thereto without departing from the broader spirit and scope of implementations of the disclosure as set forth in the following claims. The specification and drawings are, accordingly, to be regarded in an illustrative sense rather than a restrictive sense.

What is claimed is:

1. A method for providing mirror speech entrainment (MSE)-based speech therapy comprising:
obtaining a voice sample of a patient, the voice sample comprising audio of the patient speaking;
synthesizing, using the voice sample, audio of a target text read in a voice matching the patient's own voice;
presenting the target text to the patient via a computing device;
instructing the patient to read the target text aloud in synchronization with the synthesized audio;
playing the synthesized audio to the patient;
recording the patient's speech output during the synchronization;
measuring a synchronicity of the patient's speech output relative to the synthesized audio;
determining, based on the synchronicity measure, a complexity for a subsequent target text; and
generating the subsequent target text based on the determined complexity.

2. The method of claim 1, further comprising:
obtaining a video sample of the patient, the video sample comprising a recording of the patient's face while speaking;
synthesizing, using the video sample, mouth movements of the patient producing the target text, the mouth movements being synchronized with the synthesized audio; and
displaying the synthesized mouth movements to the patient via a display of the computing device during the synchronization.

3. The method of claim 2, wherein synthesizing the mouth movements comprises generating a synthesized depiction focused on the patient's mouth and perioral region.

4. The method of claim 1, wherein playing the synthesized audio comprises progressively attenuating an amplitude of the synthesized audio across successive repetitions of the target text, such that the patient's speech transitions from being externally cued by the synthesized audio to being independently generated by the patient.

5. The method of claim 1, further comprising:
receiving a selection of an entrainment mode from a conversational mode and a melodic mode; and
synthesizing the audio of the target text in accordance with the selected entrainment mode, wherein the conversational mode produces the synthesized audio with naturalistic prosody and variable stress, and the melodic mode produces the synthesized audio with exaggerated pitch contours and isochronous rhythmic timing.

6. The method of claim 1, wherein playing the synthesized audio comprises playing the synthesized audio at a playback rate that increases over the course of therapy, such that temporal demand on the patient's synchronization increases progressively.

7. The method of claim 1, further comprising:
receiving, via a clinician interface, one or more linguistic targets specifying characteristics of the target text; and
generating the target text in accordance with the one or more linguistic targets.

8. The method of claim 1, further comprising:
receiving a patient text preference; and
generating the target text based on the patient text preference.

9. The method of claim 1, further comprising:
receiving a synchronicity measure from a prior therapy session of the patient; and
selecting the target text based on the synchronicity measure from the prior therapy session.

10. The method of claim 9, wherein determining the complexity for the subsequent target text is further based on synchronicity measures accumulated across a plurality of prior therapy sessions.

11. The method of claim 1, further comprising generating a report of the therapy session comprising the synchronicity measure and the determined complexity, and transmitting the report to a clinician.

12. A computing device for providing mirror speech entrainment (MSE)-based speech therapy, the computing device comprising:
a processor;
a display;
a speaker; and
a non-transitory computer-readable medium storing instructions executable by the processor to:
obtain a voice sample of a patient, the voice sample comprising audio of the patient speaking;
synthesize, using the voice sample, audio of a target text read in a voice matching the patient's own voice;
present the target text to the patient via the display;
instruct the patient to read the target text aloud in synchronization with the synthesized audio;
play the synthesized audio to the patient via the speaker;
record the patient's speech output during the synchronization;
measure a synchronicity of the patient's speech output relative to the synthesized audio;
determine, based on the synchronicity measure, a complexity for a subsequent target text; and
generate the subsequent target text based on the determined complexity.

13. The computing device of claim 12, further comprising a camera, wherein the instructions are further executable to:
obtain a video sample of the patient, the video sample comprising a recording of the patient's face while speaking, via the camera;
synthesize, using the video sample, mouth movements of the patient producing the target text, the mouth movements being synchronized with the synthesized audio; and
display the synthesized mouth movements to the patient via the display during the synchronization.

14. The computing device of claim 13, wherein synthesizing the mouth movements comprises generating a synthesized depiction focused on the patient's mouth and perioral region.

15. The computing device of claim 12, wherein the instructions are further executable to play the synthesized audio via the speaker at a playback rate that increases over the course of therapy, such that temporal demand on the patient's synchronization increases progressively.

16. The computing device of claim 12, wherein the instructions are further executable to:
receive a selection of an entrainment mode from a conversational mode and a melodic mode; and
synthesize the audio of the target text in accordance with the selected entrainment mode, wherein the conversational mode produces the synthesized audio with naturalistic prosody and variable stress, and the melodic mode produces the synthesized audio with exaggerated pitch contours and isochronous rhythmic timing.

17. The computing device of claim 12, wherein the instructions are further executable to generate a report of the therapy session comprising the synchronicity measure and the determined complexity, and transmit the report to a clinician.

18. A non-transitory computer-readable medium storing instructions executable to:
obtain a voice sample of a patient, the voice sample comprising audio of the patient speaking;
synthesize, using the voice sample, audio of a target text read in a voice matching the patient's own voice;
present the target text to the patient via a computing device;
instruct the patient to read the target text aloud in synchronization with the synthesized audio;
play the synthesized audio to the patient;
record the patient's speech output during the synchronization;
measure a synchronicity of the patient's speech output relative to the synthesized audio;
determine, based on the synchronicity measure, a complexity for a subsequent target text; and
generate the subsequent target text based on the determined complexity.

19. The non-transitory computer-readable medium of claim 18, wherein the instructions are further executable to:
obtain a video sample of the patient, the video sample comprising a recording of the patient's face while speaking;
synthesize, using the video sample, mouth movements of the patient producing the target text, the mouth movements being synchronized with the synthesized audio; and
display the synthesized mouth movements to the patient via a display of the computing device during the synchronization.

20. The non-transitory computer-readable medium of claim 18, wherein the instructions are further executable to:
receive a selection of an entrainment mode from a conversational mode and a melodic mode; and
synthesize the audio of the target text in accordance with the selected entrainment mode, wherein the conversational mode produces the synthesized audio with naturalistic prosody and variable stress, and the melodic mode produces the synthesized audio with exaggerated pitch contours and isochronous rhythmic timing.
